(19) United States
(12) Patent Application Publication
(10) Pub. No.: US 2025/0236603 A1
(43) Pub. Date: Jul. 24, 2025

(54) SOLID FORMS OF ALECTINIB AND ALECTINIB SALTS

(71) Applicant: Synthon B.V., Nijmegen (NL)

(72) Inventor: Bohumil DYMACEK, Blansko (CZ)

(21) Appl. No.: 18/840,429

(22) PCT Filed: Feb. 21, 2023
(86) PCT No.: PCT/EP2023/054326

§ 371 Date: Aug. 21, 2024

(30) Foreign Application Priority Data
Feb. 22, 2022 (EP) 22157941.0
Feb. 22, 2022 (EP) 22157946.9
Feb. 22, 2022 (EP) 22157949.3

(51) Int. Cl. C07D 401/04

(52) U.S. Cl. CPC C07D 401/04

(57) ABSTRACT

The invention relates to Alectinib, compound of formula (1), solid forms thereof, salts thereof, solid forms of the salts and processes for preparation thereof,.

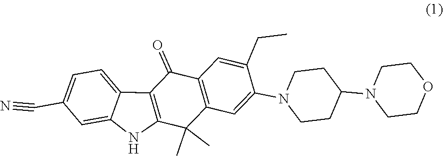 [SMILES: CCc1cc2c(cc1N1CCC(N3CCOCC3)CC1)C(C)(C)c1[nH]c3cc(C#N)ccc3c1C2=O]

The invention relates to solid forms of Alectinib or Alectinib acetate salt or Alectinib salt with fumaric acid or Alectinib salt with tartaric acid or Alectinib salt with maleic acid or

Alectinib methane sulfonate salt or Alectinib ethane sulfonate salt.

### BACKGROUND OF THE PRESENT INVENTION

This invention relates to Alectinib, compound of formula (1), solid forms thereof, salts thereof, solid forms of the salts and processes for preparation thereof:

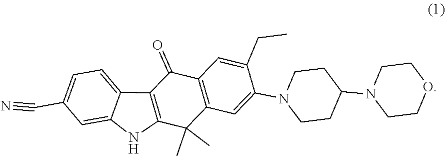 [SMILES: CCc1cc2c(cc1N1CCC(N3CCOCC3)CC1)C(C)(C)c1[nH]c3cc(C#N)ccc3c1C2=O]

Alectinib, 9-Ethyl-6,6-dimethyl-8-[4-(morpholin-4-yl) piperidin-1-yl]-11-oxo-6,11-dihydro-5H-benzo[b]carbazole-3-carbonitrile, is an ALK inhibitor. Alectinib was launched for the treatment of treatment ALK-positive, locally advanced or metastatic non-small cell lung cancer.

Alectinib was first disclosed in WO2010143664 application. WO2015163447 application describes HCl salt of Alectinib and solid forms thereof. Several salts of alectinib and solid forms thereof are described in CZ31293 utility model.

There is still a need to prepare Alectinib salts and solid forms thereof having improved solubility, crystallinity and stability in comparison with Alectinib salts solid forms disclosed in the prior art.

### BRIEF DESCRIPTION OF THE INVENTION

The presented invention relates to solid forms of Alectinib or Alectinib acetate salt or Alectinib salt with fumaric acid or Alectinib salt with tartaric acid or Alectinib salt with maleic acid or Alectinib methane sulfonate salt or Alectinib ethane sulfonate salt.

### BRIEF DESCRIPTION OF THE DRAWINGS

FIG. 1 depicts the X-Ray Powder Diffractogram (XRPD) of Alectinib acetic acid salt, Form 2, prepared according to Example 1.

FIG. 2 depicts the DSC pattern of Alectinib acetic acid salt, Form 2, prepared according to Example 1.

FIG. 3 depicts the TGA pattern of Alectinib acetic acid salt, Form 2, prepared according to Example 1.

FIG. 4 depicts the X-Ray Powder Diffractogram (XRPD) of Alectinib acetic acid salt, Form 4, prepared according to Example 2.

FIG. 5 depicts the DSC pattern of Alectinib acetic acid salt, Form 4, prepared according to Example 2.

FIG. 6 depicts the TGA pattern of Alectinib acetic acid salt, Form 4, prepared according to Example 2.

FIG. 7 depicts the X-Ray Powder Diffractogram (XRPD) of Alectinib acetic acid salt, Form 6, prepared according to Example 3.

FIG. 8 depicts the DSC pattern of Alectinib acetic acid salt, Form 6, prepared according to Example 3.

FIG. 9 depicts the TGA pattern of Alectinib acetic acid salt, Form 6, prepared according to Example 3.

FIG. 10 depicts the X-Ray Powder Diffractogram (XRPD) of Alectinib acetic acid salt, Form 7, prepared according to Example 4.

FIG. 11 depicts the DSC pattern of Alectinib acetic acid salt, Form 7, prepared according to Example 4.

FIG. 12 depicts the TGA pattern of Alectinib acetic acid salt, Form 7, prepared according to Example 4.

FIG. 13 depicts the X-Ray Powder Diffractogram (XRPD) of Alectinib, Form III, prepared according to Example 6.

FIG. 14 depicts the X-Ray Powder Diffractogram (XRPD) of Alectinib, Form IV, prepared according to Example 7.

FIG. 15 depicts the X-Ray Powder Diffractogram (XRPD) of Alectinib, Form V, prepared according to Example 8.

FIG. 16 depicts the X-Ray Powder Diffractogram (XRPD) of Alectinib, Form VI, prepared according to Example 9.

FIG. 17 depicts the X-Ray Powder Diffractogram (XRPD) of Alectinib acetic acid salt, Form 3, prepared according to Example 2.

FIG. 18 depicts the DSC pattern of Alectinib acetic acid salt, Form 3, prepared according to Example 2.

FIG. 19 depicts the TGA pattern of Alectinib acetic acid salt, Form 3, prepared according to Example 2.

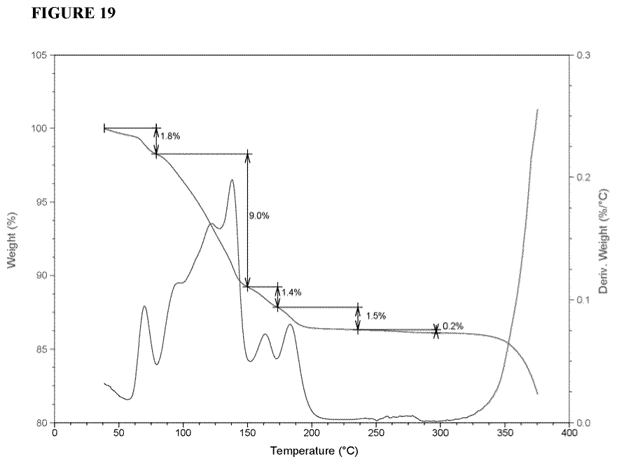

FIG. 20 depicts the X-Ray Powder Diffractogram (XRPD) of Alectinib acetic acid salt, Form 5, prepared according to Example 2.

FIG. 21 depicts NMR spectrum of Alectinib acetic acid salt, Form 2, prepared according to Example 1.

FIG. 22 depicts the X-Ray Powder Diffractogram (XRPD) of Alectinib tartarate salt, Form 8, prepared according to Example 11.

FIG. 23 depicts the DSC pattern of Alectinib tartarate salt, Form 8, prepared according to Example 11.

FIG. 24 depicts the TGA pattern of Alectinib tartarate salt, Form 8, prepared according to Example 11.

FIG. 25 depicts the X-Ray Powder Diffractogram (XRPD) of Alectinib hydrogen maleate salt, Form 1, prepared according to Example 12.

FIG. 26 depicts the DSC pattern of Alectinib hydrogen maleate salt, Form 1, prepared according to Example 12.

FIG. 27 depicts the TGA pattern of Alectinib hydrogen maleate salt, Form 1, prepared according to Example 12.

FIG. 28 depicts the X-Ray Powder Diffractogram (XRPD) of Alectinib hydrogen maleate salt, Form 2, prepared according to Example 13 or Example 14.

FIG. 29 depicts the DSC pattern of Alectinib hydrogen maleate salt, Form 2, prepared according to Example 13 or Example 14.

FIG. 30 depicts the TGA pattern of Alectinib hydrogen maleate salt, Form 2, prepared according to Example 13 or Example 14.

FIG. 31 depicts the X-Ray Powder Diffractogram (XRPD) of Alectinib fumaric acid (1:1) salt, Form 2, prepared according to Example 15.

FIG. 32 depicts the DSC pattern of Alectinib fumaric acid (1:1) salt, Form 2, prepared according to Example 15.

FIG. 33 depicts the TGA pattern of Alectinib fumaric acid (1:1) salt, Form 2, prepared according to Example 15.

FIG. 34 depicts the X-Ray Powder Diffractogram (XRPD) of Alectinib fumaric acid (1:1) salt, Form 6, prepared according to Example 16.

FIG. 35 depicts the DSC pattern of Alectinib fumaric acid (1:1) salt, Form 6, prepared according to Example 16.

FIG. 36 depicts the TGA pattern of Alectinib fumaric acid (1:1) salt, Form 6, prepared according to Example 16.

FIG. 37 depicts the X-Ray Powder Diffractogram (XRPD) of Alectinib fumaric acid (2:1) salt, Form 2, prepared according to Example 17.

FIG. 38 depicts the DSC pattern of Alectinib fumaric acid (2:1) salt, Form 2, prepared according to Example 17.

FIG. 39 depicts the TGA pattern of Alectinib fumaric acid (2:1) salt, Form 2, prepared according to Example 17.

FIG. 40 depicts NMR spectrum of Alectinib fumaric acid salt (2:1), Form 2, prepared according to Example 17.

FIG. 41 depicts NMR spectrum of Alectinib fumaric acid (1:1) salt, Form 2, prepared according to Example 15.

FIG. 42 depicts the NMR spectrum of Alectinib fumaric acid (1:1) salt, Form 6, prepared according to Example 16.

FIG. 43 depicts the NMR spectrum of Alectinib hydrogen maleate salt, Form 2, prepared according to Example 13 or Example 14.

FIG. 44 depicts the X-Ray Powder Diffractogram (XRPD) of Alectinib ethane sulfonate salt, Form 1, prepared according to Example 19.

FIG. 45 depicts the DSC pattern of Alectinib ethane sulfonate salt, Form 1, prepared according to Example 19 or Example 27.

FIG. 46 depicts the TGA pattern of Alectinib ethane sulfonate salt, Form 1, prepared according to Example 19 or Example 27.

FIG. 47 depicts the X-Ray Powder Diffractogram (XRPD) of Alectinib ethane sulfonate salt, Form 2, prepared according to Example 20.

FIG. 48 depicts the DSC pattern of Alectinib ethane sulfonate salt, Form 2, prepared according to Example 20.

FIG. 49 depicts the TGA pattern of Alectinib ethane sulfonate salt, Form 2, prepared according to Example 20.

FIG. 50 depicts the X-Ray Powder Diffractogram (XRPD) of Alectinib ethane sulfonate salt, Form 7, prepared according to Example 21.

FIG. 51 depicts the DSC pattern of Alectinib ethane sulfonate salt, Form 7, prepared according to Example 21.

FIG. 52 depicts the TGA pattern of Alectinib ethane sulfonate salt, Form 7, prepared according to Example 21.

FIG. 53 depicts the X-Ray Powder Diffractogram (XRPD) of Alectinib methane sulfonate salt, Form 3, prepared according to Example 23.

FIG. 54 depicts the DSC pattern of Alectinib methane sulfonate salt, Form 3, prepared according to Example 23.

FIG. 55 depicts the TGA pattern of Alectinib methane sulfonate salt, Form 3, prepared according to Example 23.

FIG. 56 depicts the X-Ray Powder Diffractogram (XRPD) of Alectinib methane sulfonate salt, Form 12, prepared according to Example 24.

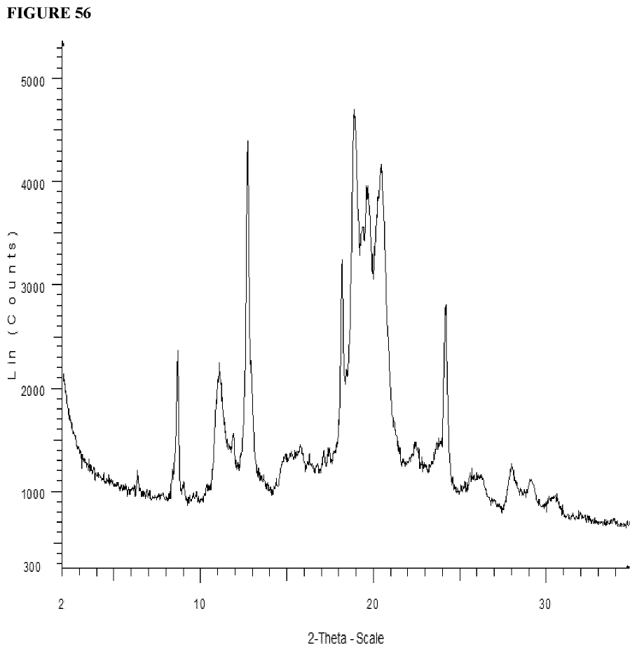

FIG. 57 depicts the DSC pattern of Alectinib methane sulfonate salt, Form 12, prepared according to Example 24.

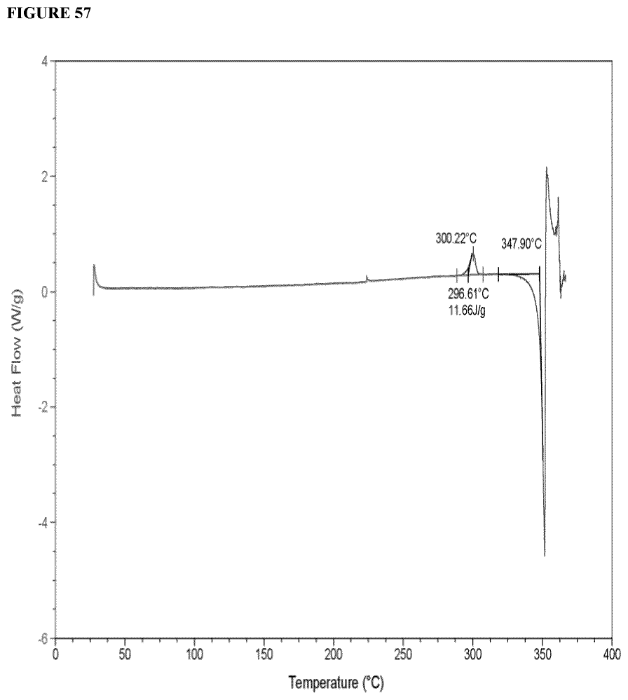

FIG. 58 depicts the TGA pattern of Alectinib methane sulfonate salt, Form 12, prepared according to Example 24.

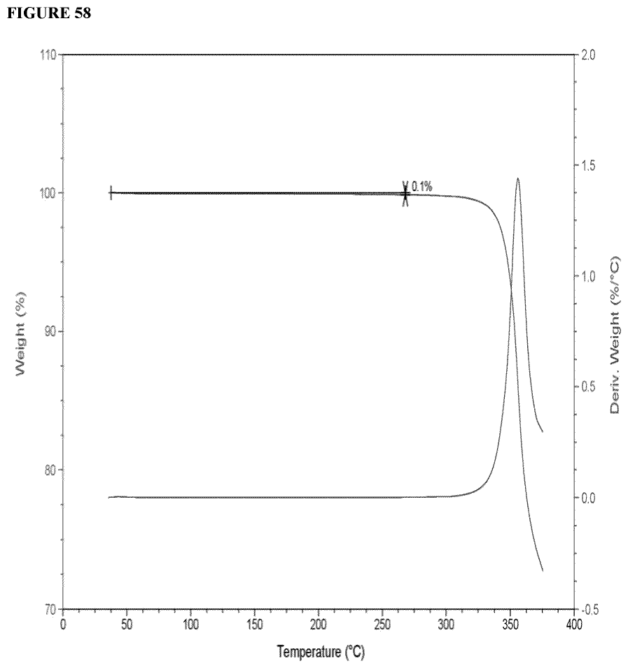

FIG. 59 depicts the X-Ray Powder Diffractogram (XRPD) of Alectinib methane sulfonate salt, Form 13, prepared according to Example 25.

FIG. 60 depicts the DSC pattern of Alectinib methane sulfonate salt, Form 13, prepared according to Example 25.

FIG. 61 depicts the TGA pattern of Alectinib methane sulfonate salt, Form 13, prepared according to Example 25.

FIG. 62 depicts the X-Ray Powder Diffractogram (XRPD) of Alectinib methane sulfonate salt, Form 14, prepared according to Example 26.

FIG. 63 depicts the DSC pattern of Alectinib methane sulfonate salt, Form 14, prepared according to Example 26.

FIG. 64 depicts the TGA pattern of Alectinib methane sulfonate salts, Form 14, prepared according to Example 26.

FIG. 65 depicts the X-Ray Powder Diffractogram (XRPD) of Alectinib methane sulfonate salt, Form 15, prepared according to Example 26.

FIG. 66 depicts the DSC pattern of Alectinib methane sulfonate salt, Form 15, prepared according to Example 26.

FIG. 67 depicts the TGA pattern of Alectinib methane sulfonate salt, Form 15, prepared according to Example 26.

FIG. 68 depicts NMR spectrum of Alectinib ethane sulfonate salt, Form 1, prepared according to Example 19.

FIG. 69 depicts NMR spectrum of Alectinib ethane sulfonate salt, Form 2, prepared according to Example 20.

FIG. 70 depicts NMR spectrum of Alectinib ethane sulfonate salt, Form 7, prepared according to Example 21.

FIG. 71 depicts NMR spectrum of Alectinib methane sulfonate salt, Form 3, prepared according to Example 23.

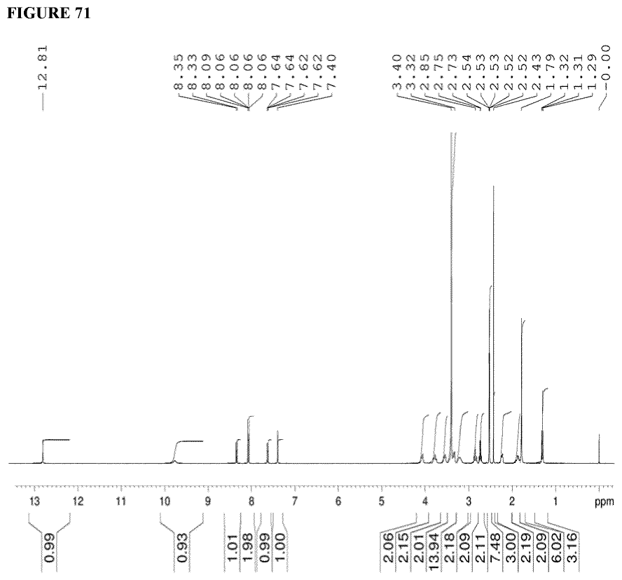

FIG. 72 depicts NMR spectrum of Alectinib methane sulfonate salt, Form 12, prepared according to Example 24.

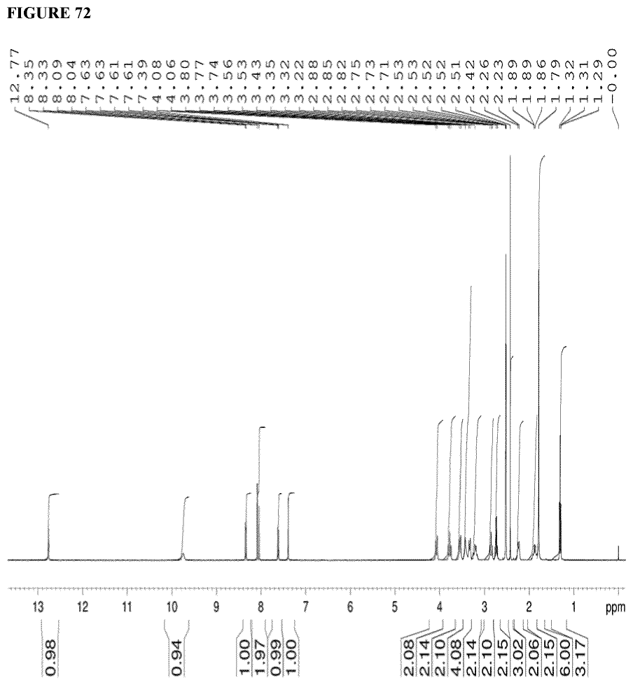

FIG. 73 depicts NMR spectrum of Alectinib methane sulfonate salt, Form 15, prepared according to Example 26.

FIG. 74 depicts NMR spectrum of Alectinib methane sulfonate salt, Form 14, prepared according to Example 26.

### DETAILED DESCRIPTION OF THE INVENTION

The presented invention relates to Alectinib acetate salt, solid forms thereof (Form 2, Form 3, Form 4, Form5, Form 6, Form 7) and processes for preparation thereof.

The solid form of Alectinib acetate salt, Form 2, can be characterized by XRPD pattern having 2θ values 10.7°, 11.0°, 13.1° and 19.9° degrees 2 theta (±0.2 degrees 2 theta). The solid Form 2 can be also characterized by XRPD pattern having 2θ values 8.3°, 8.8°, 10.7°, 11.0°, 13.1° and 19.9° degrees 2 theta (±0.2 degrees 2 theta). The solid form can be further characterized by XRPD pattern described in the following table:

| Angle (2θ) | Intensity (%) |
|---|---|
| 4.9 | 12.4 |
| 5.2 | 22.6 |
| 7.4 | 19.1 |
| 8.3 | 26.7 |
| 8.8 | 36.7 |
| 9.3 | 15.7 |
| 10.7 | 63.2 |
| 11.0 | 67.3 |
| 11.9 | 30.2 |
| 12.3 | 36.2 |
| 13.1 | 76.4 |
| 13.9 | 14.0 |
| 14.4 | 15.4 |
| 14.8 | 19.6 |
| 15.4 | 22.9 |
| 15.7 | 41.1 |
| 16.4 | 19.1 |
| 16.6 | 20.8 |
| 17.0 | 16.3 |
| 17.5 | 26.1 |
| 18.5 | 35.2 |
| 18.9 | 67.6 |
| 19.1 | 61.6 |
| 19.6 | 66.8 |
| 19.9 | 100.0 |
| 20.6 | 59.5 |
| 21.5 | 37.8 |
| 22.1 | 32.7 |
| 22.4 | 31.9 |
| 23.8 | 19.0 |
| 24.0 | 17.8 |
| 25.3 | 16.8 |
| 25.6 | 11.9 |
| 26.7 | 13.8 |
| 27.6 | 17.3 |
| 28.9 | 20.6 |
| 29.8 | 21.2 |
| 30.7 | 10.9 |
| 31.5 | 9.6 |
| 32.2 | 9.6 |
| 33.0 | 10.2 |
| 33.8 | 7.3 |

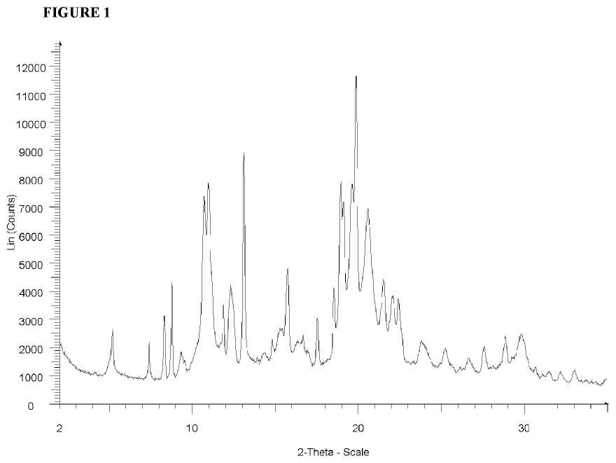

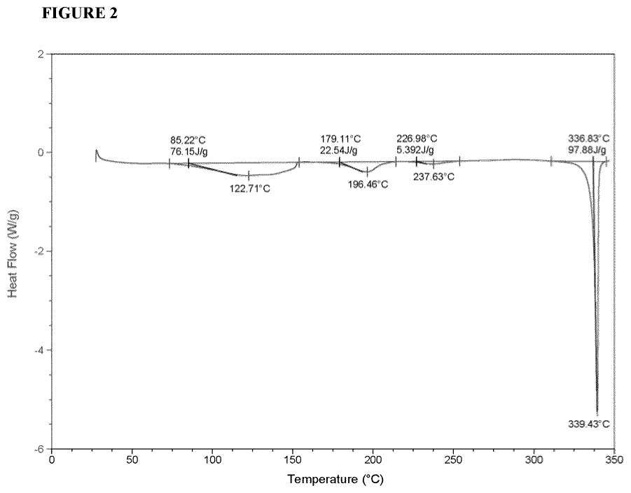

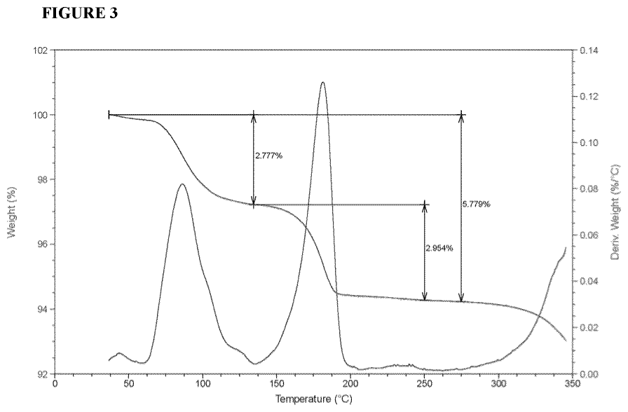

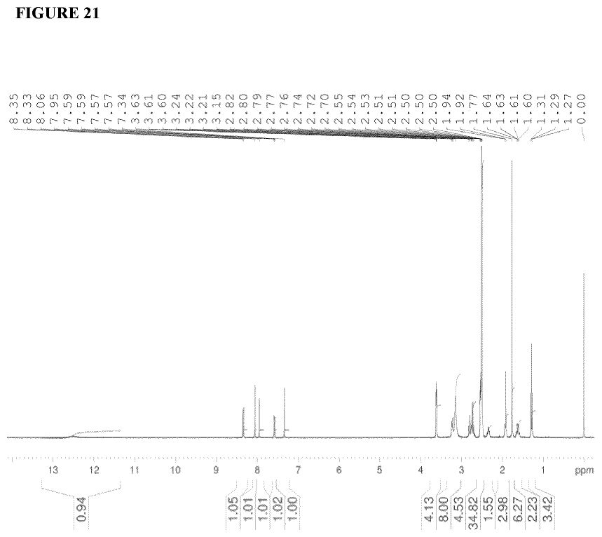

The solid Form 2 can be also characterized by XRPD pattern depicted in FIG. 1. The solid form can be further characterized by DSC pattern depicted in FIG. 2. The solid form can be further characterized by TGA pattern depicted in FIG. 3. The solid form can be also characterized by NMR spectrum depicted in FIG. 21.

The solid Form 2 of Alectinib acetate salt can be prepared by a process comprising:
a. Mixing Alectinib with a mixture of 2-butanone and acetic acid;
b. Isolating he solid form.

The concentration of Alectinib in 2-butanone can be between 0.06 g/ml and 0.1 g/ml.

The molar ratio between Alectinib and acetic acid can be between 1:18 and 1:22. Alectinib is mixed with 2-butanone and acetic acid. The mixture was stirred at room temperature (20-25° C.) for between 20 and 60 minutes to obtain a suspension. Suspension was filtered off and dried at 20-25° C. in vacuum (1-10 kPa) for between 1 and 10 days to provide solid Form 2 of Alectinib acetate.

The solid form of Alectinib acetate salt, Form 3, can be characterized by XRPD pattern having 2θ values 9.3°, 19.2° and 20.0° degrees 2 theta (±0.2 degrees 2 theta). The solid Form 3 can be also characterized by XRPD pattern having 2θ values 5.0°, 9.3°, 9.9°, 19.2° and 20.0° degrees 2 theta (±0.2 degrees 2 theta). The solid form can be further characterized by XRPD pattern described in the following table:

| Angle (2θ) | Intensity (%) |
|---|---|
| 5.0 | 28.5 |
| 5.4 | 9.5 |
| 7.2 | 16.6 |
| 8.2 | 13.7 |
| 8.6 | 13.9 |
| 8.7 | 13.3 |
| 9.3 | 100.0 |
| 9.6 | 16.7 |
| 9.9 | 40.1 |
| 10.2 | 17.7 |
| 10.6 | 17.2 |
| 10.9 | 15.3 |
| 11.3 | 12.6 |
| 11.9 | 12.0 |
| 12.2 | 13.7 |
| 12.6 | 14.3 |
| 13.1 | 17.4 |
| 13.7 | 34.4 |
| 14.0 | 19.2 |
| 14.4 | 23.9 |
| 14.9 | 11.4 |
| 15.4 | 18.6 |
| 15.6 | 18.3 |
| 16.4 | 15.8 |
| 16.7 | 18.6 |
| 17.5 | 13.1 |
| 17.9 | 17.3 |
| 18.1 | 14.8 |
| 18.5 | 17.7 |
| 19.2 | 73.8 |
| 20.0 | 94.8 |
| 20.4 | 26.9 |
| 21.0 | 20.6 |
| 21.5 | 29.4 |
| 21.7 | 25.2 |
| 22.2 | 19.1 |
| 22.7 | 14.0 |
| 23.3 | 12.2 |
| 23.9 | 18.2 |
| 24.6 | 21.9 |
| 25.4 | 14.4 |
| 25.7 | 11.8 |
| 26.6 | 9.1 |
| 27.1 | 15.0 |
| 29.3 | 18.7 |
| 30.0 | 8.8 |
| 31.1 | 8.8 |
| 32.6 | 7.2 |

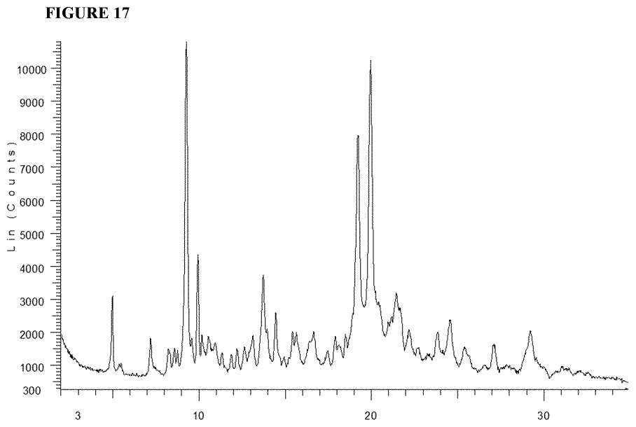

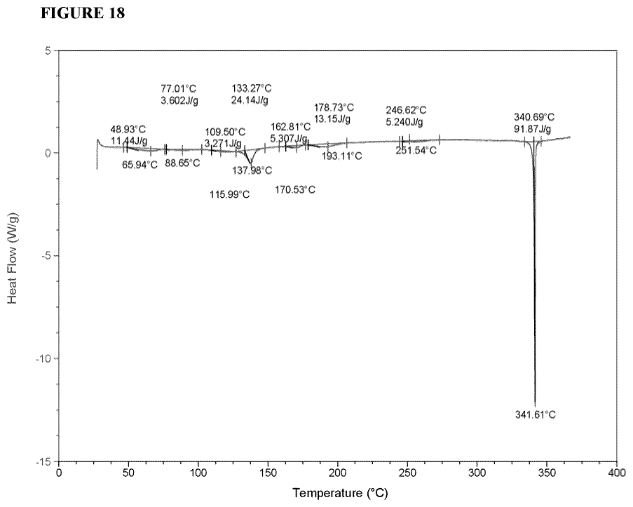

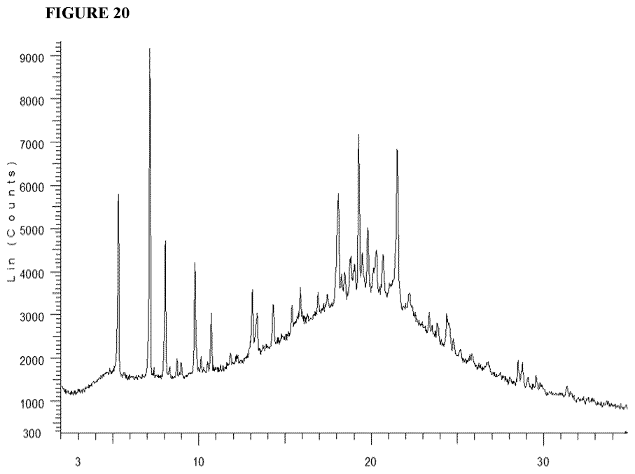

The solid Form 3 can be also characterized by XRPD pattern depicted in FIG. 17. The solid form can be further characterized by DSC pattern depicted in FIG. 18. The solid form can be further characterized by TGA pattern depicted in FIG. 20.

The solid Form 3 of Alectinib acetate salt can be prepared by a process comprising drying solid Form 5 of Alectinib acetate in vacuum dryer at a temperature between 20-25° C. for between 10 and 15 hours.

The solid Form 4 of Alectinib acetate salt, can be characterized by XRPD pattern having 2θ values 7.5°, 10.4° and 22.8° degrees 2 theta (±0.2 degrees 2 theta). The solid Form 4 can be also characterized by XRPD pattern having 2θ values 7.5°, 10.4°, 16.4°, 18.4° and 22.8° degrees 2 theta (±0.2 degrees 2 theta). The solid form can be further characterized by XRPD pattern described in the following table:

| Angle (2θ) | Intensity (%) |
|---|---|
| 6.8 | 7.0 |
| 7.5 | 100.0 |
| 8.7 | 6.0 |
| 8.9 | 11.8 |
| 9.2 | 2.3 |
| 9.9 | 7.9 |
| 10.4 | 47.7 |
| 11.8 | 3.8 |
| 12.1 | 8.3 |
| 12.5 | 2.0 |
| 13.7 | 11.9 |
| 14.1 | 7.5 |
| 14.9 | 13.6 |
| 15.6 | 8.2 |
| 16.4 | 19.2 |
| 16.9 | 5.0 |
| 17.2 | 15.8 |
| 17.5 | 7.2 |
| 18.0 | 10.6 |
| 18.4 | 12.4 |
| 18.8 | 3.4 |
| 19.7 | 25.1 |
| 20.4 | 19.8 |
| 20.9 | 9.2 |
| 21.3 | 6.5 |
| 21.6 | 11.1 |
| 22.3 | 7.4 |
| 22.8 | 55.3 |
| 23.4 | 12.8 |
| 23.7 | 21.0 |
| 24.1 | 8.8 |
| 24.4 | 15.9 |
| 24.7 | 5.4 |
| 25.4 | 2.4 |
| 26.1 | 4.7 |
| 26.4 | 4.5 |
| 26.8 | 5.6 |
| 27.2 | 8.0 |
| 27.6 | 4.9 |
| 28.0 | 4.7 |
| 28.3 | 7.3 |
| 28.8 | 2.7 |
| 29.9 | 6.7 |
| 30.4 | 3.7 |
| 31.0 | 4.1 |
| 31.5 | 5.5 |
| 32.5 | 1.9 |
| 33.1 | 3.0 |
| 33.7 | 2.3 |
| 34.1 | 2.5 |
| 34.8 | 2.3 |

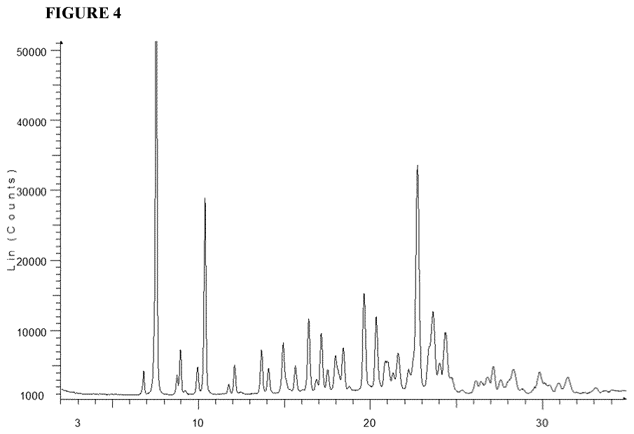

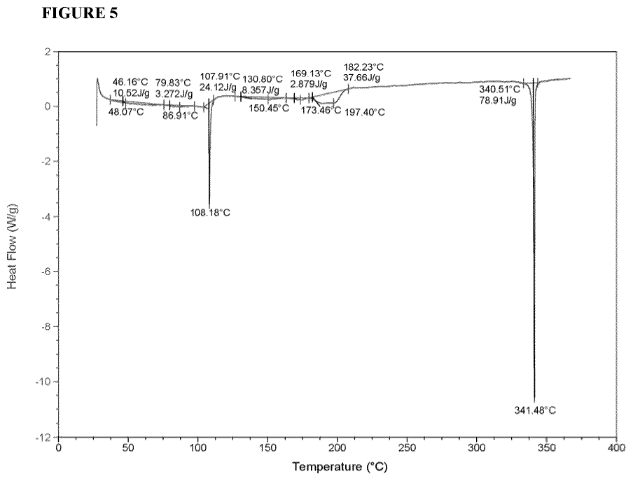

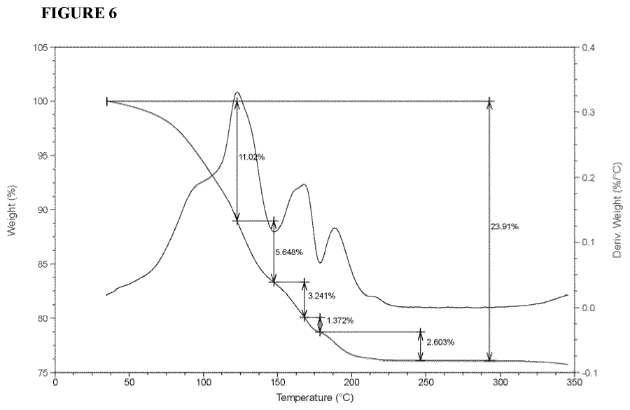

The solid Form 4 can be also characterized by XRPD pattern depicted in FIG. 4. The solid form can be further characterized by DSC pattern depicted in FIG. 5. The solid form can be further characterized by TGA pattern depicted in FIG. 6.

The solid Form 4 of Alectinib acetate salt can be prepared by a process comprising:
a. Mixing Alectinib with a mixture of 2-butanone and acetic acid;
b. Isolating the solid form.

The concentration of Alectinib in 2-butanone can be between 0.06 g/ml and 0.1 g/ml. The molar ratio between Alectinib and acetic acid can be between 1:17 and 1:20. Alectinib is mixed with a mixture of 2-butanone and acetic acid. The mixture is sonicated for between 1 and 5 minutes and the mixture was stirred at room temperature (20-25° C.) for between 20 and 60 minutes to obtain a suspension. A part of suspension is filtered off and analyzed using XRPD. The solid corresponds to solid Form 5 of Alectinib acetate salt. The suspension is stirred for between 5 and 12 hours and filtered off. Obtained solid is dried on air at room temperature (20-25° C.) for between 5 and 15 hours to provide solid Form 4 of Alectinib acetic acid salt. In the case the filtered off solid is dried in vacuum at room temperature for between 8 and 15 hours, solid Form 3 of Alectinib acetate salt is obtained.

The solid Form 5 of Alectinib acetate salt can be characterized by XRPD pattern having 2θ values 5.3°, 7.1°, 8.0° and 21.5° degrees 2 theta (±0.2 degrees 2 theta). The solid Form 5 can be also characterized by XRPD pattern having 2θ values 5.3°, 7.1°, 8.0°, 9.8°, 13.1°, 18.1° and 21.5° degrees 2 theta (±0.2 degrees 2 theta). The solid form can be further characterized by XRPD pattern described in the following table:

| Angle (2θ) | Intensity (%) |
|---|---|
| 4.8 | 19.1 |
| 5.3 | 63.1 |
| 7.1 | 100.0 |
| 7.4 | 19.2 |
| 8.0 | 51.3 |
| 8.3 | 19.5 |
| 8.7 | 21.4 |
| 9.0 | 20.6 |
| 9.8 | 45.8 |
| 10.1 | 22.0 |
| 10.5 | 20.7 |
| 10.7 | 33.0 |
| 11.8 | 22.9 |
| 12.2 | 22.5 |
| 13.1 | 39.0 |
| 13.4 | 33.1 |
| 14.3 | 35.2 |
| 15.4 | 35.1 |
| 15.9 | 39.5 |
| 16.9 | 38.4 |
| 17.4 | 37.9 |
| 18.0 | 47.9 |
| 18.1 | 63.3 |
| 18.3 | 42.7 |
| 18.5 | 43.4 |
| 18.8 | 46.7 |
| 19.1 | 45.4 |
| 19.3 | 78.3 |
| 19.5 | 48.1 |
| 19.8 | 54.5 |
| 20.3 | 49.0 |
| 20.7 | 47.8 |
| 21.5 | 74.5 |
| 22.2 | 38.2 |
| 23.4 | 33.1 |
| 23.5 | 29.8 |
| 23.9 | 29.6 |
| 24.4 | 32.8 |
| 24.5 | 30.5 |
| 24.8 | 26.5 |
| 25.2 | 23.9 |
| 25.9 | 22.8 |
| 26.8 | 20.7 |
| 28.1 | 16.8 |
| 28.5 | 21.2 |
| 28.8 | 20.5 |
| 29.1 | 16.8 |
| 29.6 | 17.2 |
| 29.8 | 15.4 |
| 31.4 | 14.4 |
| 34.5 | 10.1 |

The solid Form 5 can be also characterized by XRPD pattern depicted in FIG. 20. The solid Form 5 of Alectinib acetate can be prepared by a process described for solid Form 4 of Alectinib acetate.

The solid Form 6 of Alectinib acetate salt can be characterized by XRPD pattern having 2θ values 8.8°, 13.2° and 19.9° degrees 2 theta (±0.2 degrees 2 theta). The solid Form 6 can be also characterized by XRPD pattern having 2θ values 8.8°, 10.0°, 13.2°, 15.8°, 19.1° and 19.9° degrees 2 theta (±0.2 degrees 2 theta). The solid form can be further characterized by XRPD pattern described in the following table:

| Angle (2θ) | Intensity (%) |
|---|---|
| 7.4 | 18.5 |
| 8.4 | 24.9 |
| 8.8 | 41.2 |
| 9.1 | 5.2 |
| 9.6 | 7.6 |
| 10.0 | 23.8 |
| 10.2 | 18.6 |
| 10.6 | 14.0 |
| 11.9 | 25.0 |
| 12.3 | 10.1 |
| 13.2 | 65.4 |
| 14.0 | 7.7 |
| 14.9 | 11.1 |
| 15.8 | 35.1 |
| 16.8 | 8.1 |
| 17.0 | 9.7 |
| 17.6 | 26.4 |
| 18.2 | 15.3 |
| 18.6 | 39.3 |
| 19.1 | 54.0 |
| 19.6 | 13.6 |
| 19.9 | 100.0 |
| 20.3 | 19.1 |
| 20.7 | 17.6 |
| 21.3 | 15.9 |
| 21.6 | 36.6 |
| 22.3 | 14.0 |
| 22.5 | 22.4 |
| 23.1 | 7.3 |
| 23.9 | 11.8 |
| 24.5 | 5.6 |
| 25.3 | 7.8 |
| 26.2 | 7.9 |
| 26.7 | 10.8 |
| 27.7 | 14.7 |
| 29.0 | 16.2 |
| 29.3 | 9.8 |
| 30.1 | 8.0 |
| 30.7 | 7.1 |
| 31.2 | 5.8 |
| 31.6 | 7.5 |
| 32.3 | 7.0 |
| 33.1 | 7.9 |

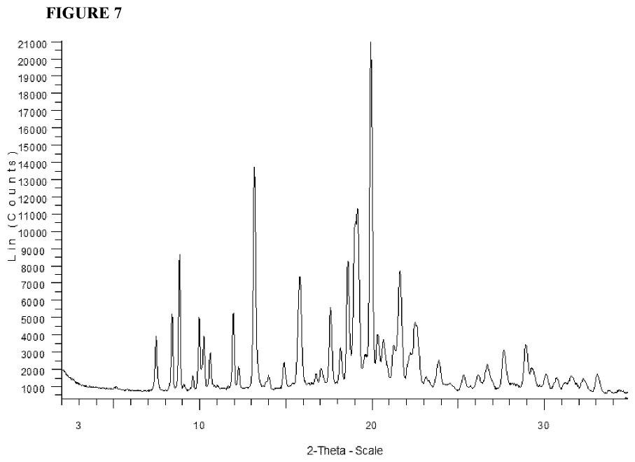

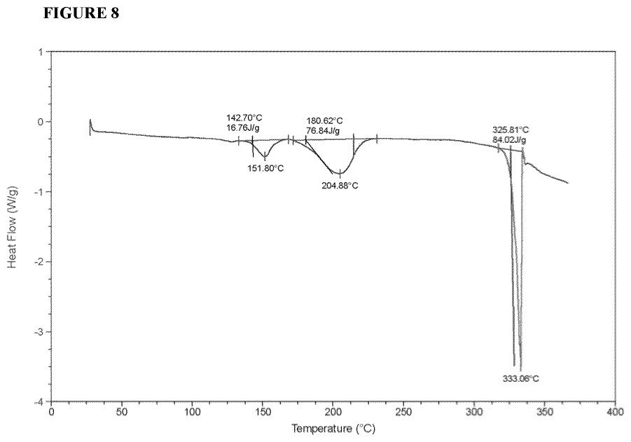

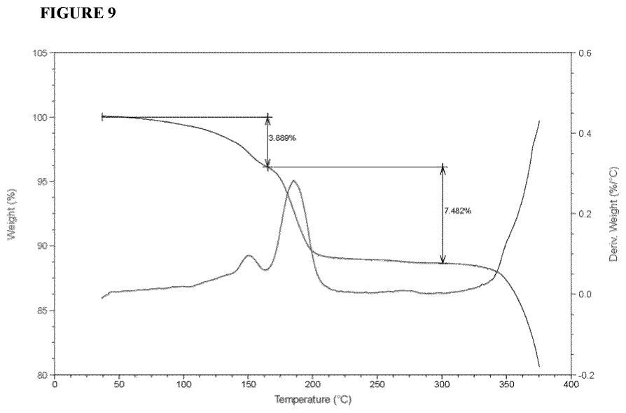

The solid Form 6 can be also characterized by XRPD pattern depicted in FIG. 7. The solid form can be further characterized by DSC pattern depicted in FIG. 8. The solid form can be further characterized by TGA pattern depicted in FIG. 9.

The solid Form 6 of Alectinib acetate salt can be prepared by a process comprising:
a. Mixing Alectinib with a mixture of 2-butanone and acetic acid;
b. Isolating the solid form.

The concentration of Alectinib in 2-butanone can be between 0.06 g/ml and 0.1 g/ml. The molar ratio between Alectinib and acetic acid can be between 1:18 and 1:25. Alectinib is mixed with the mixture 2-butanone and acetic acid. The mixture is sonicated for between 1 and 5 minutes to obtain a suspension. The solid in suspension is XRPD analyzed. It corresponds to solid Form 5 of Alectinib acetate. The mixture is heated at between 45° C. and 55° C. for between 5 and 30 minutes. Then mixture was cooled to between 20° C. and 25° C. and filtrated. Obtained solid is dried on air for between 3 and 6 days to provide solid Form 6 of Alectinib acetate salt.

The solid Form 7 of Alectinib acetate salt, can be characterized by XRPD pattern having 2θ values 9.9°, 10.2° and 20.3° degrees 2 theta (±0.2 degrees 2 theta). The solid Form 7 can be also characterized by XRPD pattern having 2θ values 9.9°, 10.2°, 10.5°, 18.1° and 20.3° degrees 2 theta (±0.2 degrees 2 theta). The solid form can be further characterized by XRPD pattern described in the following table:

| Angle (2θ) | Intensity (%) |
|---|---|
| 5.1 | 3.8 |
| 7.2 | 3.4 |
| 8.4 | 2.9 |
| 8.5 | 3.1 |
| 8.7 | 3.3 |
| 9.0 | 7.3 |
| 9.9 | 100.0 |
| 10.2 | 67.0 |
| 10.5 | 41.8 |
| 10.9 | 4.8 |
| 11.5 | 3.2 |
| 11.9 | 6.5 |
| 13.0 | 18.2 |
| 13.8 | 10.4 |
| 14.4 | 6.0 |
| 15.3 | 7.4 |
| 15.6 | 22.8 |
| 16.2 | 8.0 |
| 16.5 | 8.6 |
| 16.9 | 11.9 |
| 17.6 | 6.0 |
| 18.1 | 46.6 |
| 18.5 | 26.6 |
| 19.0 | 51.0 |
| 19.5 | 22.1 |
| 19.8 | 21.6 |
| 20.3 | 57.9 |
| 20.5 | 17.8 |
| 20.8 | 11.6 |
| 21.2 | 43.7 |
| 21.5 | 37.0 |
| 22.3 | 8.3 |
| 23.1 | 15.0 |
| 23.9 | 14.8 |
| 24.2 | 8.5 |
| 24.4 | 10.6 |
| 25.5 | 5.8 |
| 26.1 | 12.4 |
| 26.5 | 7.9 |
| 27.3 | 6.3 |
| 27.5 | 9.0 |
| 28.3 | 7.3 |
| 29.2 | 7.5 |
| 29.5 | 4.2 |
| 30.1 | 14.2 |
| 30.5 | 9.1 |
| 31.0 | 5.2 |
| 31.5 | 4.9 |
| 31.8 | 8.1 |
| 32.3 | 4.5 |
| 32.9 | 3.9 |
| 33.7 | 4.5 |
| 34.4 | 4.0 |
| 34.6 | 3.5 |

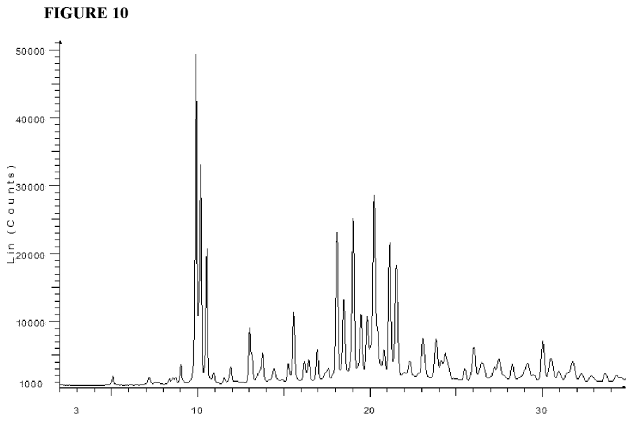

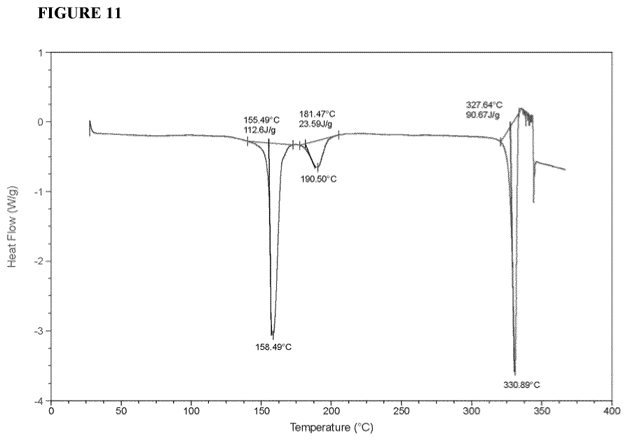

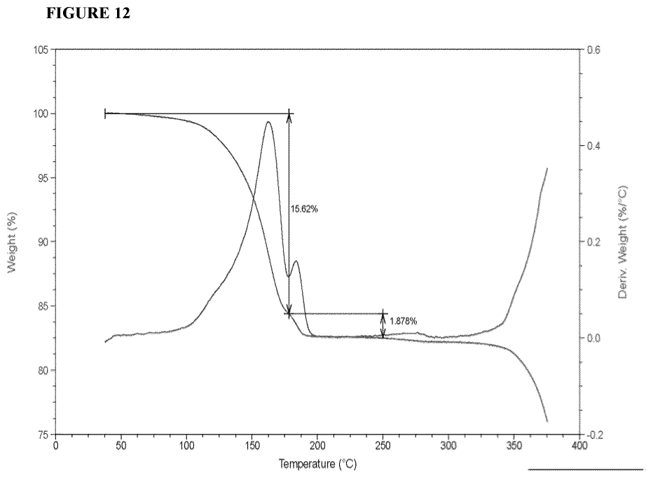

The solid Form 7 can be also characterized by XRPD pattern depicted in FIG. 10. The solid form can be further characterized by DSC pattern depicted in FIG. 11. The solid form can be further characterized by TGA pattern depicted in FIG. 12.

The solid Form 7 of Alectinib acetate salt can be prepared by a process comprising:
a. Mixing Alectinib with a mixture of 2-butanone and acetic acid;
b. Isolating the solid form.

The concentration of Alectinib in 2-butanone can be between 0.06 g/ml and 0.1 g/ml. The molar ratio between Alectinib and acetic acid can be between 1:18 and 1:25. Alectinib is mixed with the mixture of 2-butanone and acetic acid. The mixture is sonicated for between 1 and 5 minutes to obtain a suspension. The solid from the suspension is XRPD analyzed. It corresponds to solid Form 5 of Alectinib acetate. The mixture is heated at a temperature between 45° C. and 55° C. for between 10 and 60 minutes. Obtained suspension is cooled to between 20° C. and 25° C. and filtered off. Obtained solid was dried on air for between 3 and 10 days to provide solid Form 7 of Alectinib acetate.

The invention also relates to solid forms of Alectinib, Form III, Form IV, Form V and Form VI.

Alectinib solid Form III can be characterized by XRPD pattern having 2θ values 7.2°, 9.9° and 20.4° degrees 2 theta (±0.2 degrees 2 theta). Alecinib solid Form III can be also characterized by XRPD pattern having 2θ values 7.2°, 9.9°, 14.0°, 18.7° and 20.4° degrees 2 theta (±0.2 degrees 2 theta). The solid form can be further characterized by XRPD pattern described in the following table:

| Angle (2- | Intensity |
|---|---|
| Theta) | (%) |
| 4.7 | 30.9 |
| 7.2 | 58.7 |
| 9.3 | 33.2 |
| 9.6 | 24.2 |
| 9.9 | 61.1 |
| 10.0 | 22.9 |
| 12.6 | 26.7 |
| 13.4 | 29.3 |
| 14.0 | 53.4 |
| 16.7 | 22.1 |
| 17.2 | 29.9 |
| 17.6 | 30.4 |
| 17.8 | 53.3 |
| 18.7 | 96.9 |
| 20.2 | 79.9 |
| 20.4 | 100.0 |
| 21.1 | 61.7 |
| 23.1 | 26.6 |

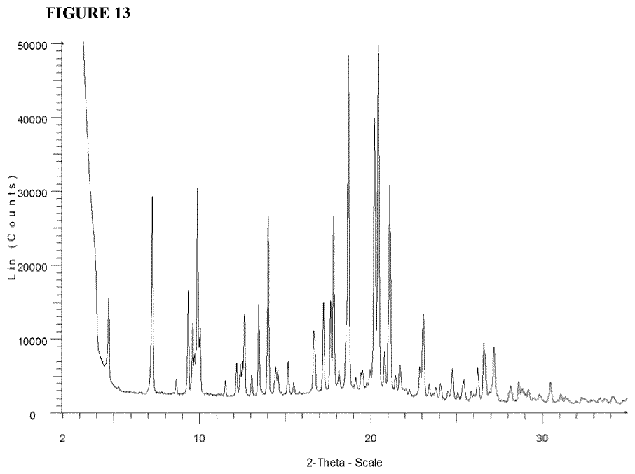

The solid Form III can be also characterized by XRPD pattern depicted in FIG. 13. The solid Form III of Alectinib can be prepared by a process comprising:
a. Mixing Alectinib with acetone or acetonitrile to obtain a mixture;
b. Heating the mixture to reflux;
c. Isolating the solid form.

The concentration of Alectinib in acetone or acetonitrile can be between 0.004 g/ml and 0.015 g/ml. Alectinib is mixed with either acetone or acetonitrile. The mixture is heated to reflux and stirred at this temperature for between 2 and 5 hours. The mixture is cooled to a temperature between 20-25° C. and filtered off. Obtained solid can be optionally dried to provide solid Form III of Alectinib.

Alectinib solid Form IV can be characterized by XRPD pattern having 2θ values 9.1°, 10.1° and 21.1° degrees 2 theta (±0.2 degrees 2 theta). Alectinib solid Form IV can be also characterized by XRPD pattern having 2θ values 9.1°, 10.1°, 18.4° and 21.1° degrees 2 theta (±0.2 degrees 2 theta). The solid form can be further characterized by XRPD pattern described in the following table:

| Angle (2- | Intensity |
|---|---|
| Theta) | (%) |
| 9.1 | 43.9 |
| 10.1 | 100.0 |
| 12.4 | 5.3 |
| 13.1 | 5.3 |
| 17.8 | 5.5 |
| 18.4 | 21.2 |
| 19.0 | 11.9 |
| 19.2 | 9.3 |
| 19.8 | 10.1 |
| 20.3 | 21.0 |
| 21.1 | 28.8 |

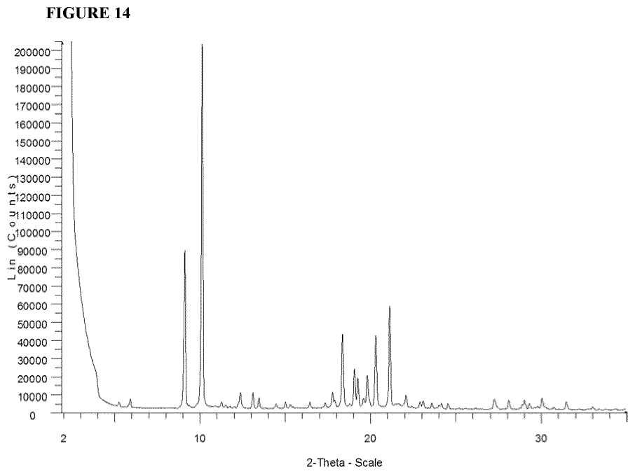

The solid Form IV can be also characterized by XRPD pattern depicted in FIG. 14.

The solid Form IV of Alectinib can be prepared by a process comprising:
a. Mixing Alectinib with 2-propanol to obtain a mixture;
b. Heating the mixture at reflux;
c. Isolating the solid form.

The concentration of Alectinib in 2-propanol can be between 0.005 g/ml and 0.009 g/ml. Alectinib is mixed with 2-propanol to obtain a mixture. The mixture is heated to reflux and stirred at this temperature for between 10 and 60 minutes. The isolating step c. can be performed either by cooling the mixture to a temperature between 20-25° C. or to the mixture an antisolvent (solvent that poorly dissolves Alectinib) is added and the mixture is stirred at a temperature between −5° C. and 5° C. for between 10 and 60 minutes. Obtained suspension is filtered off and obtained solid can be optionally dried to provide solid Form IV of Alectinib. Alectinib solid Form V can be characterized by XRPD pattern having 2θ values 7.9°, 15.9° and 20.3° degrees 2 theta (±0.2 degrees 2 theta). Alectinib solid Form V can be also characterized by XRPD pattern having 2θ values 7.9°, 14.0°, 15.9°, 17.7°, 20.3° and 24.0° degrees 2 theta (±0.2 degrees 2 theta). The solid form can be further characterized by XRPD pattern described in the following table:

| Angle (2- | Intensity |
|---|---|
| Theta) | (%) |
| 7.9 | 78.3 |
| 9.5 | 25.3 |
| 11.7 | 21.5 |
| 14.0 | 42.1 |
| 14.9 | 35.4 |
| 15.1 | 18.9 |
| 15.9 | 73.9 |
| 16.9 | 20.0 |
| 17.3 | 41.4 |
| 17.7 | 44.3 |
| 19.4 | 24.1 |
| 19.8 | 28.7 |
| 20.0 | 37.1 |
| 20.3 | 100.0 |
| 22.1 | 39.9 |
| 22.4 | 17.5 |
| 23.9 | 47.9 |
| 26.1 | 13.6 |
| 26.3 | 13.9 |
| 27.2 | 23.4 |

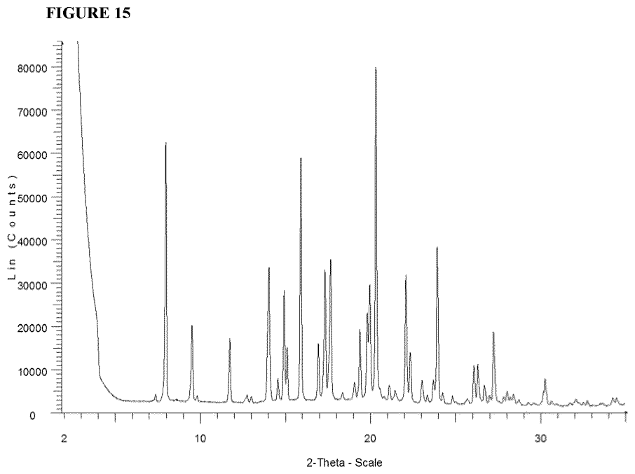

The solid Form V can be also characterized by XRPD pattern depicted in FIG. 15.

The solid Form V of Alectinib can be prepared by a process comprising:
a. Mixing Alectinib with methanol to obtain a mixture;
b. Heating the mixture to reflux;
c. Isolating the solid form.

The concentration of Alectinib in methanol can be between 0.03 g/ml and 0.06 g/ml. The mixture is heated to reflux and stirred for 5 minutes. The mixture is cooled to 20-25° C. Obtained solid is filtered off and obtained solid can be optionally dried at 20-25° C. to provide solid Form V of Alectinib.

Alectinib solid Form VI can be characterized by XRPD pattern having 2θ values 8.3°, 9.6° and 20.3° degrees 2 theta (±0.2 degrees 2 theta). Alecinib solid Form VI can be also characterized by XRPD pattern having 2θ values 8.3°, 9.6°, 17.0°, 18.4° and 20.3° degrees 2 theta (±0.2 degrees 2 theta). The solid form can be further characterized by XRPD pattern described in the following table:

| Angle (2- | Intensity |
|---|---|
| Theta) | (%) |
| 8.3 | 46.4 |
| 9.3 | 15.4 |
| 9.6 | 57.8 |
| 12.6 | 9.7 |
| 13.1 | 6.4 |
| 13.3 | 6.3 |
| 14.6 | 11.5 |
| 15.2 | 5.3 |
| 15.4 | 7.8 |
| 16.6 | 5.5 |
| 17.0 | 30.8 |
| 18.0 | 19.5 |
| 18.4 | 29.9 |
| 18.9 | 8.6 |
| 19.3 | 19.2 |
| 19.9 | 25.5 |
| 20.3 | 100.0 |
| 20.8 | 8.2 |
| 21.2 | 6.3 |
| 21.5 | 7.4 |
| 21.7 | 15.1 |
| 22.9 | 6.9 |
| 23.3 | 6.6 |
| 24.8 | 7.0 |
| 25.0 | 6.4 |
| 25.3 | 7.4 |
| 26.7 | 6.3 |
| 27.5 | 5.4 |
| 28.8 | 7.8 |
| 29.2 | 7.6 |
| 29.3 | 9.4 |
| 31.8 | 5.5 |

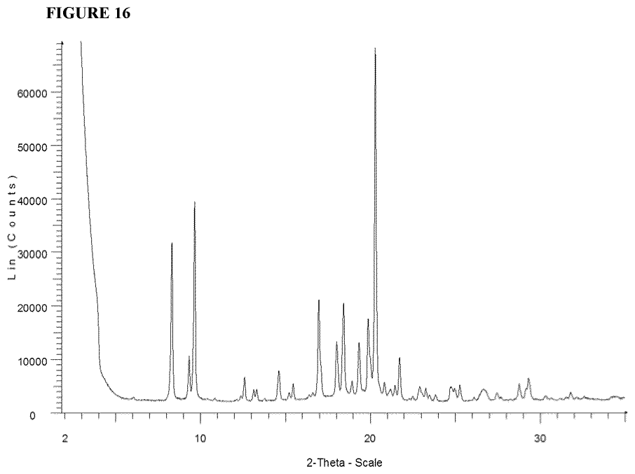

The solid Form VI can be also characterized by XRPD pattern depicted in FIG. 16.

The solid Form VI of Alectinib can be prepared by a process comprising:
a. Mixing Alectinib with 1,4-dioxane to obtain a mixture;
b. Heating the mixture to reflux;
c. Isolating the form.

The concentration of Alectinib in 1,4-dioxane can be between 0.004 g/ml and 0.009 g/ml. Alectinib is mixed with 1,4-dioxane. The mixture is heated to reflux. The mixture is cooled to a temperature between 20-25° C. and stirred at this temperature for between 5 minutes. The suspension is filtered off and obtained solid can be optionally dried to provide solid Form VI of Alectinib.

The presented invention also relates to Alectinib tartrate salt (salt with tartaric acid), a solid form thereof (Form 8) and to a process for preparing thereof.

The Form 8 can be characterized by XRPD pattern having 2θ values 5.2°, 12.3° and 19.0° degrees 2 theta (±0.2 degrees 2 theta). The solid Form 8 can be also characterized by XRPD pattern having 2θ values 5.2°, 6.1°, 9.3°, 12.3° and 19.0° degrees 2 theta (±0.2 degrees 2 theta). The solid form can be further characterized by XRPD pattern described in the following table:

| Angle (2θ) | Intensity (%) |
|---|---|
| 5.2 | 32.7 |
| 6.1 | 29.9 |
| 7.2 | 18.2 |
| 8.4 | 12.2 |
| 9.3 | 30.7 |
| 11.2 | 39.2 |
| 11.7 | 25.1 |
| 12.3 | 75.6 |
| 12.6 | 23.4 |
| 14.2 | 21.9 |
| 14.5 | 25.9 |
| 15.2 | 30.2 |
| 18.2 | 27.2 |
| 19.0 | 100.0 |
| 19.6 | 45.1 |
| 20.2 | 22.8 |
| 21.0 | 31.2 |
| 22.0 | 38.9 |
| 23.3 | 42.0 |
| 23.8 | 23.4 |
| 24.2 | 17.4 |
| 24.9 | 15.6 |
| 25.7 | 15.2 |
| 27.7 | 11.2 |
| 28.6 | 13.6 |
| 29.6 | 13.6 |
| 30.2 | 17.7 |

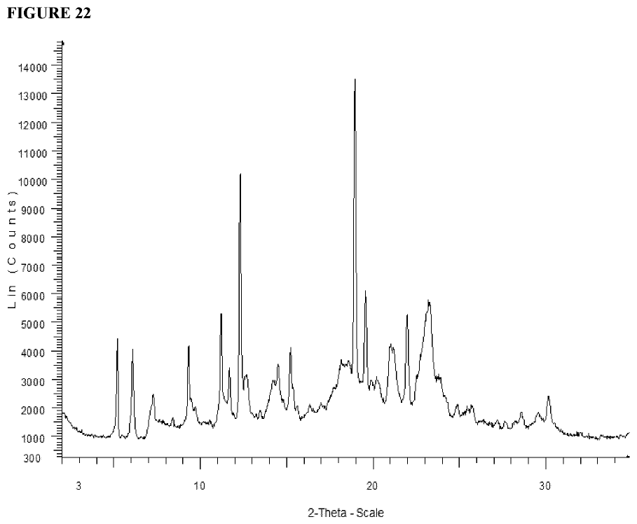

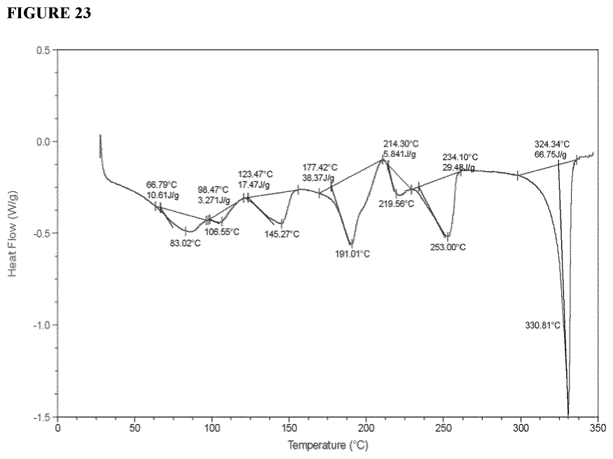

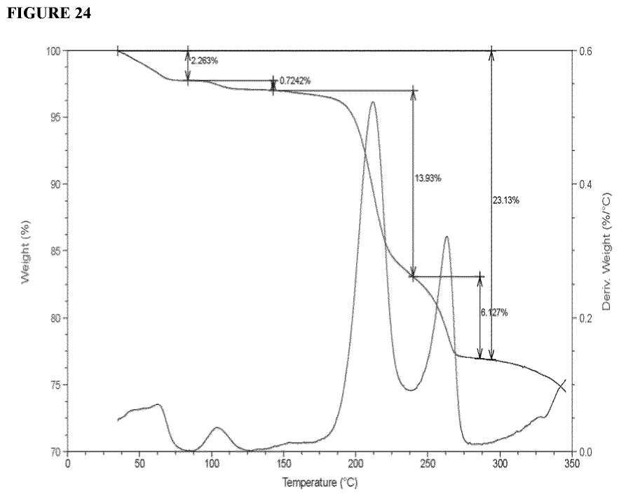

The solid Form 8 can be also characterized by XRPD pattern depicted in FIG. 22. The solid form can be further characterized by DSC pattern depicted in FIG. 23. The solid form can be further characterized by TGA pattern depicted in FIG. 24.

The solid Form 8 of Alectinib tartrate salt can be prepared by a process comprising:
a. Mixing Alectinib and L-(+)-tartaric acid with water;
b. Mixing the mixture for between 1 and 5 hours at 20-25° C.;
c. Isolating the solid form.

The concentration of Alectinib in water can be between 0.1 g/ml and 0.3 g/ml. The molar ratio between Alectinib and tartaric acid can be between 1:1 and 1:1.2. Alectinib and L-(+)-tartaric acid is mixed with water. The mixture is stirred for between 1 and 5 hours at 20-25° C. The mixture is filtered. Obtained solid is mixed with acetone. The volume ratio between acetone and water used in step a. can be between 1:5 and 1:15. The mixture is stirred for between 5 and 15 hours at 20-25° C. and filtered. Obtained solid is dried on air at 20-25° C. for between 0.5 and 3 days to provide solid Form 8 of Alectinib tartrate salt.

The presented invention further relates to Alectinib hydrogen maleate salt, solid forms thereof (Form 1, Form 2) and processes for preparing thereof.

The solid form, Form 1, of Alectinib hydrogen maleate salt can be characterized by XRPD pattern having 2θ values 12.2°, 19.2° and 20.4° degrees 2 theta (±0.2 degrees 2 theta). The solid Form 1 can be also characterized by XRPD pattern having 2θ values 11.5°, 12.2°, 15.8°, 19.2° and 20.4° degrees 2 theta (±0.2 degrees 2 theta). The solid form can be further characterized by XRPD pattern described in the following table:

| Angle (2θ) | Intensity (%) |
|---|---|
| 6.2 | 2.6 |
| 7.8 | 1.8 |
| 9.0 | 2.8 |
| 10.2 | 7.6 |
| 10.8 | 13.5 |
| 11.2 | 11.3 |
| 11.5 | 17.2 |
| 12.2 | 18.4 |
| 13.0 | 2.8 |
| 14.3 | 2.4 |
| 15.4 | 3.2 |
| 15.6 | 4.3 |
| 15.8 | 17.7 |
| 16.5 | 2.3 |
| 17.0 | 1.5 |
| 18.0 | 3.3 |
| 18.4 | 4.8 |
| 18.7 | 2.2 |
| 19.2 | 77.8 |
| 19.8 | 22.1 |
| 20.4 | 100.0 |
| 20.8 | 4.3 |
| 21.7 | 7.5 |
| 22.1 | 8.4 |
| 22.5 | 3.3 |
| 23.1 | 2.7 |
| 23.6 | 10.7 |
| 24.3 | 2.4 |
| 24.8 | 1.8 |
| 25.0 | 1.5 |
| 25.4 | 2.9 |
| 26.1 | 3.0 |
| 26.6 | 1.8 |
| 27.1 | 1.6 |
| 27.5 | 2.8 |
| 28.4 | 2.4 |
| 28.6 | 11.5 |
| 29.1 | 2.4 |
| 29.4 | 8.1 |
| 30.2 | 2.7 |
| 30.4 | 2.9 |
| 30.9 | 4.2 |
| 31.4 | 1.8 |
| 31.8 | 3.4 |
| 32.6 | 1.8 |
| 33.0 | 1.0 |
| 33.5 | 1.3 |
| 34.6 | 1.3 |

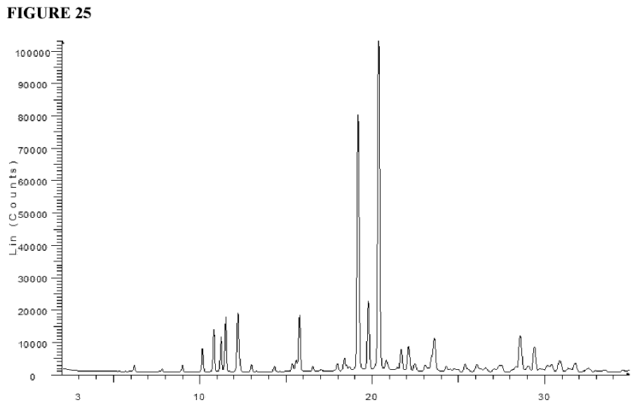

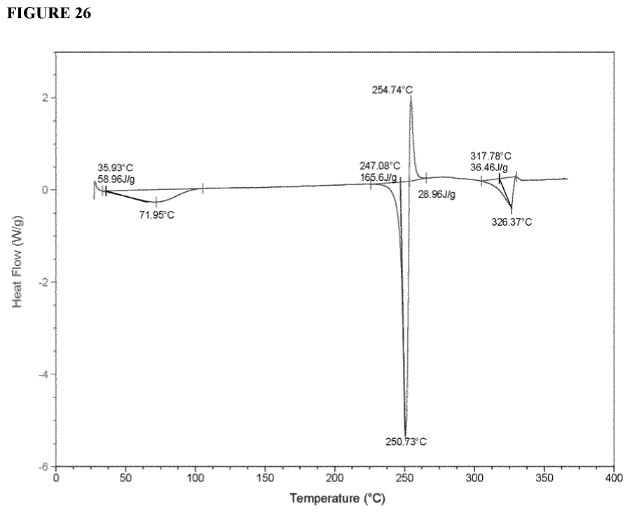

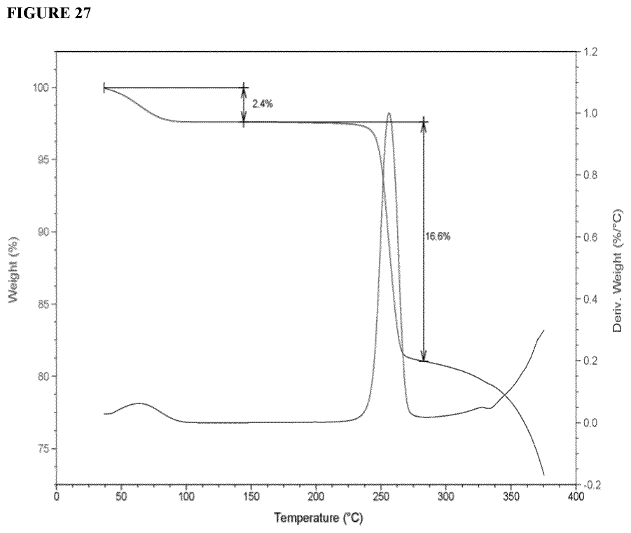

The solid Form 1 can be also characterized by XRPD pattern depicted in FIG. 25. The solid form can be further characterized by DSC pattern depicted in FIG. 26. The solid form can be further characterized by TGA pattern depicted in FIG. 27.

The solid Form 1 of Alectinib hydrogen maleate salt can be prepared by a process comprising:
a. Mixing of Alectinib with a mixture of 2-butanone, acetic acid and water;
b. Adding of maleic acid;
c. Isolating the solid form.

The concentration of Alectinib in the mixture 2-butanone, acetic acid and water can be between 0.04 g/ml and 0.07 g/ml. The volume ratio between 2-butanone and acetic acid can be between 5.5:1 and 6.5:1. The volume ratio between 2-butanone and water can be between 3:1 and 4:1. Molar ratio between Alectinib and maleic acid can be between 1:1 and 1:1.1. Alectinib is mixed with a mixture of 2-butanone, acetic acid and water. To the mixture, maleic acid is added. Maleic acid is added preferably in a form of a solution in a suitable solvent for example in water. The mixture is stirred at room temperature (20-25° C.) for between 5 and 12 hours. Obtain suspension is filtered off and optionally dried on air to provide solid Form 1 of Alectinib hydrogen maleate salt.

The solid Form 2 of Alectinib hydrogen maleate salt can be characterized by XRPD pattern having 2θ values 7.7°, 18.4° and 23.1° degrees 2 theta (±0.2 degrees 2 theta). The solid Form 2 can be also characterized by XRPD pattern having 2θ values 7.7°, 15.2°, 17.7°, 18.4°, 22.7° and 23.1° degrees 2 theta (±0.2 degrees 2 theta). The solid form can be further characterized by XRPD pattern described in the following table:

| Angle (2θ) | Intensity (%) |
|---|---|
| 7.2 | 19.0 |
| 7.7 | 63.0 |
| 9.3 | 7.3 |
| 9.5 | 4.9 |
| 10.1 | 8.5 |
| 11.6 | 42.0 |
| 11.9 | 8.6 |
| 12.3 | 10.3 |
| 12.6 | 3.7 |
| 14.4 | 7.0 |
| 15.0 | 9.6 |
| 15.2 | 38.0 |
| 15.4 | 17.5 |
| 15.7 | 16.8 |
| 16.3 | 23.4 |
| 17.0 | 7.9 |
| 17.3 | 10.6 |
| 17.5 | 17.9 |
| 17.7 | 33.2 |
| 18.1 | 15.5 |
| 18.4 | 94.6 |
| 18.5 | 24.9 |
| 19.1 | 3.9 |
| 20.5 | 4.7 |
| 20.9 | 6.3 |
| 21.2 | 9.4 |
| 21.4 | 21.4 |
| 21.6 | 21.9 |
| 22.4 | 24.8 |
| 22.7 | 76.3 |
| 23.1 | 100.0 |
| 23.4 | 30.5 |
| 23.8 | 18.5 |
| 24.2 | 6.7 |
| 24.5 | 11.4 |
| 24.8 | 33.6 |
| 25.6 | 10.1 |
| 26.1 | 12.1 |
| 27.3 | 8.1 |
| 27.7 | 4.4 |
| 28.5 | 8.7 |
| 29.1 | 22.8 |
| 29.3 | 8.1 |
| 30.2 | 11.0 |
| 30.5 | 9.0 |
| 30.9 | 9.2 |
| 31.8 | 7.9 |
| 31.9 | 6.8 |
| 32.3 | 6.4 |
| 32.9 | 6.7 |
| 33.5 | 4.4 |
| 33.7 | 4.4 |
| 34.3 | 11.0 |
| 15.4 | 20.5 |
| 15.9 | 10.3 |

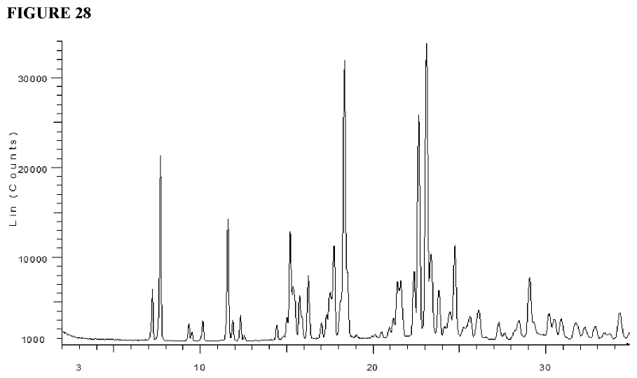

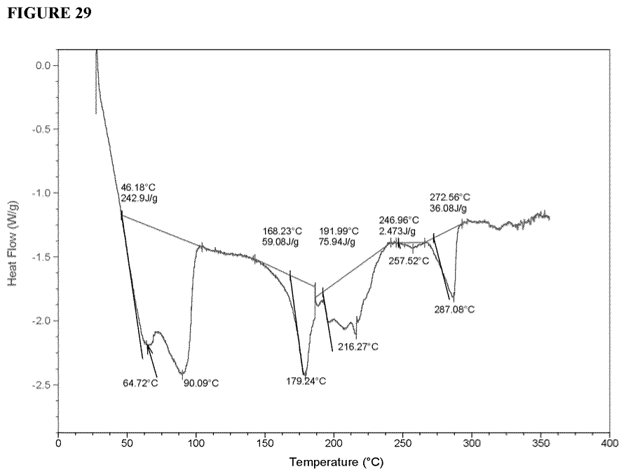

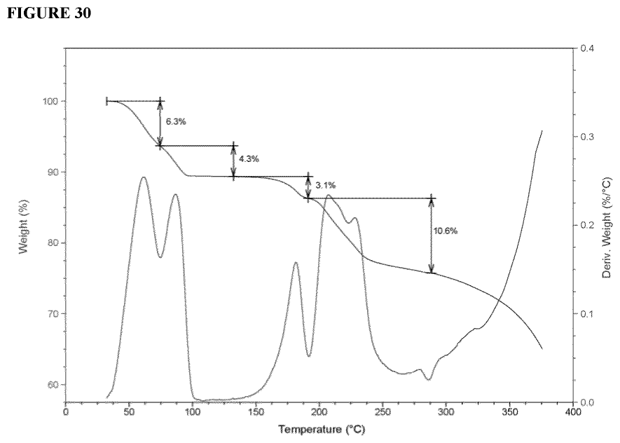

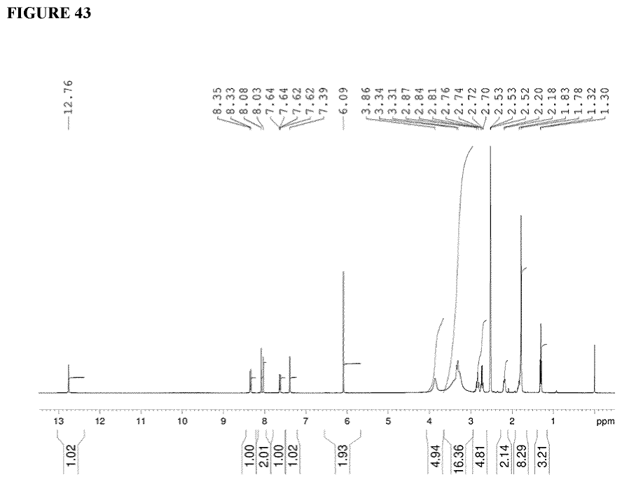

The solid Form 2 can be also characterized by XRPD pattern depicted in FIG. 28. The solid form can be further characterized by DSC pattern depicted in FIG. 29. The solid form can be further characterized by TGA pattern depicted in FIG. 30. The solid form can be also characterized by NMR spectrum depicted in FIG. 43.

The solid Form 2 of Alectinib hydrogen maleate salt can be prepared by a process comprising drying of Form 1 of Alectinib hydrogen maleate under vacuum. Form 1 is dried under vacuum (1-15 kPa) at room temperature (20-25° C.) for between 5 and 14 hours to provide solid Form 2 of Alectinib salt with maleic acid.

The presented invention also relates to solid forms (Form 2, Form 6) of Alectinib fumaric acid (1:1) salt.

The solid Form 2 of Alectinib fumaric acid (1:1) salt can be characterized by XRPD pattern having 2θ values 13.0°, 18.9° and 21.7° degrees 2 theta (±0.2 degrees 2 theta). The solid Form 2 can be also characterized by XRPD pattern having 2θ values 13.0°, 18.9°, 19.2°, 20.1° and 21.7° degrees 2 theta (±0.2 degrees 2 theta). The solid form can be further characterized by XRPD pattern described in the following table:

| Angle (2θ) | Intensity (%) |
|---|---|
| 6.8 | 36.2 |
| 8.2 | 26.7 |
| 8.4 | 28.3 |
| 9.4 | 16.5 |
| 11.3 | 36.8 |
| 11.6 | 34.7 |
| 12.5 | 3.9 |
| 13.0 | 100.0 |
| 13.7 | 16.7 |
| 15.1 | 5.0 |
| 16.1 | 2.4 |
| 16.6 | 8.1 |
| 16.8 | 11.7 |
| 17.7 | 2.6 |
| 18.5 | 15.3 |
| 18.9 | 97.3 |
| 19.2 | 65.8 |
| 20.1 | 56.8 |
| 20.5 | 12.3 |
| 20.8 | 43.7 |
| 21.7 | 91.3 |
| 22.0 | 21.8 |
| 22.6 | 6.8 |
| 23.1 | 13.2 |
| 23.4 | 10.2 |
| 24.0 | 10.8 |
| 24.7 | 3.2 |
| 25.0 | 5.1 |
| 25.4 | 12.5 |
| 26.1 | 9.8 |
| 26.7 | 21.5 |
| 27.3 | 5.6 |
| 27.6 | 6.7 |
| 28.2 | 12.3 |
| 28.4 | 13.8 |
| 29.4 | 6.6 |
| 30.4 | 10.1 |
| 31.0 | 4.4 |
| 31.7 | 3.5 |
| 32.2 | 4.7 |
| 32.8 | 4.6 |
| 33.5 | 2.6 |
| 34.2 | 2.8 |
| 34.4 | 3.0 |

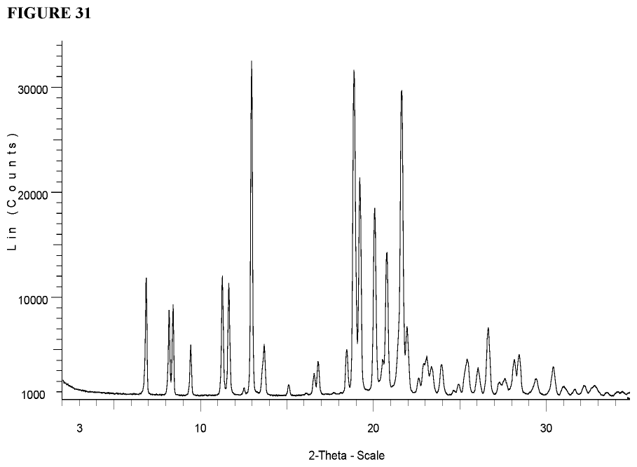

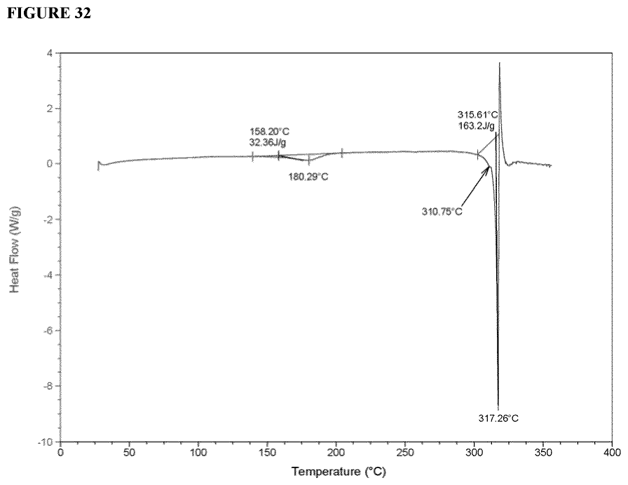

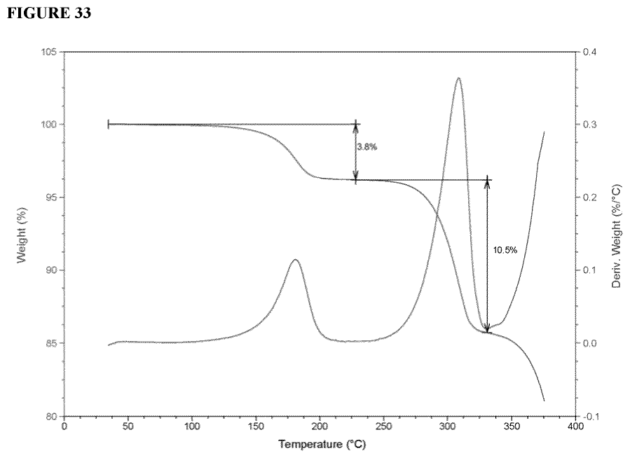

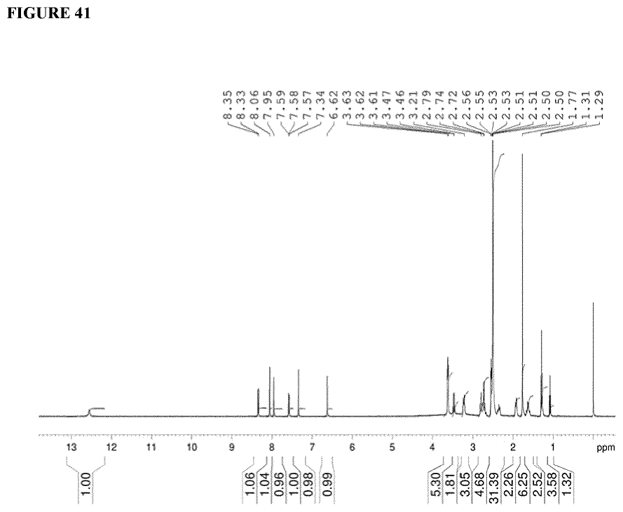

The solid Form 2 can be also characterized by XRPD pattern depicted in FIG. 31. The solid form can be further characterized by DSC pattern depicted in FIG. 32. The solid form can be further characterized by TGA pattern depicted in FIG. 33. The solid form can be also characterized by NMR spectrum depicted in FIG. 41.

The solid Form 2 of Alectinib fumaric acid (1:1) salt can be prepared by a process comprising:
a. Mixing Alectinib with a mixture of 2-butanone, water and acetic acid to obtain a mixture;
b. Adding the mixture into solution of fumaric acid in ethanol and water;
c. Isolating a solid form;
d. Mixing the solid form with ethanol and stirring at a temperature between 65° C. and 75° C. for between 20 and 60 minutes;
e. Stirring at 20-25° C. for between 10 and 30 hours;
f. Isolating the solid Form 2.

The volume ratio between 2-butanone and water can be between 2.4:1 and 3.2:1. The volume ratio between 2-butanone and acetic acid can be between 1:2.8 and 1:3.5. The concentration of Alectinib in the mixture 2-butanone, water and acetic acid can be between 0.03 and 0.07 g/ml. The concentration of fumaric acid in the mixture of ethanol and water can be between 0.005 g/ml and 0.009 g/ml. The molar ratio between Alectinib and fumaric acid can be between 1:1 and 1:1.2. Alectinib is mixed with the mixture of 2-butanone, water and acetic acid. Obtained mixture is added, preferably portion wise, for example in 2 or 3 or 4 or 5 or 6 or 7 or 8, more preferably dropwise, to a mixture of fumaric acid in ethanol and water. The mixture is stirred at 20-25° C. for between 5 and 15 hours to obtain a suspension. The suspension was filtered off to obtain a solid form of Alectinib fumaric acid salt. Obtained solid form is mixed with ethanol. The concentration of the solid form in ethanol can be between 0.05-0.2 g/ml. The mixture is heated to 60-80° C. for between 30 and 60 minutes and then stirred at 20-25° C. for between 10 and 30 hours. The mixture is filtered and obtained solid was dried on air at 20-25° C. for between 5 and 15 hours to provide solid Form 2 of Alectinib fumaric acid salt.

The solid Form 6 of Alectinib fumaric acid (1:1) salt can be characterized by XRPD pattern having 2θ values 7.4°, 12.7°, 18.9°and 23.0° degrees 2 theta (±0.2 degrees 2 theta).

The solid Form 6 can be also characterized by XRPD pattern having 2θ values 7.4°, 12.7°, 14.8°, 18.9°, 20.5° and 23.0° degrees 2 theta (=0.2 degrees 2 theta). The solid form can be further characterized by XRPD pattern described in the following table:

| Angle (2θ) | Intensity (%) |
|---|---|
| 7.4 | 78.5 |
| 7.7 | 11.2 |
| 8.5 | 10.9 |
| 8.8 | 10.6 |
| 11.5 | 4.3 |
| 12.7 | 60.3 |
| 13.1 | 33.5 |
| 13.2 | 33.9 |
| 13.6 | 5.7 |
| 13.9 | 7.5 |
| 14.2 | 34.9 |
| 14.8 | 36.0 |
| 15.2 | 7.1 |
| 15.5 | 10.6 |
| 17.6 | 29.8 |
| 18.0 | 18.6 |
| 18.1 | 22.6 |
| 18.3 | 15.8 |
| 18.4 | 13.2 |
| 18.9 | 60.4 |
| 19.2 | 20.4 |
| 19.5 | 14.9 |
| 19.8 | 5.3 |
| 20.5 | 48.4 |
| 21.0 | 13.3 |
| 21.4 | 13.1 |
| 21.9 | 7.4 |
| 22.3 | 30.6 |
| 23.0 | 100.0 |
| 23.4 | 12.9 |
| 23.6 | 68.8 |
| 24.0 | 11.3 |
| 24.3 | 28.0 |
| 24.6 | 16.7 |
| 24.8 | 16.4 |
| 25.1 | 13.2 |
| 25.6 | 12.1 |
| 26.4 | 9.0 |
| 26.6 | 9.5 |
| 27.0 | 9.1 |
| 27.3 | 10.3 |
| 27.6 | 6.8 |
| 28.1 | 4.4 |
| 28.5 | 6.0 |
| 29.4 | 8.0 |
| 29.6 | 15.3 |
| 29.9 | 10.1 |
| 30.2 | 7.9 |
| 30.4 | 7.8 |
| 31.0 | 6.5 |
| 33.1 | 8.2 |
| 31.5 | 6.3 |
| 32.8 | 10.2 |
| 33.6 | 4.2 |
| 33.9 | 5.4 |

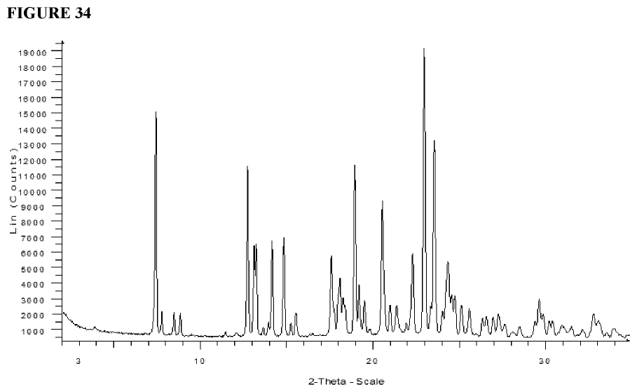

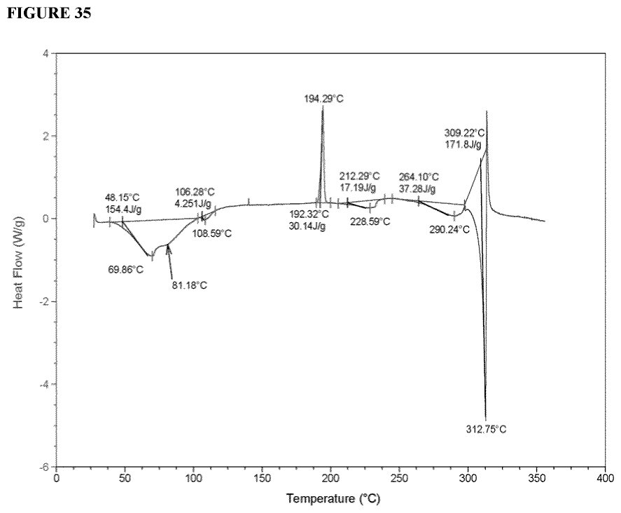

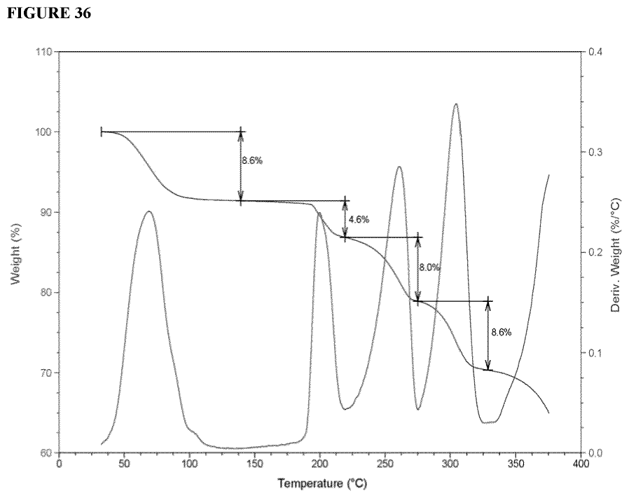

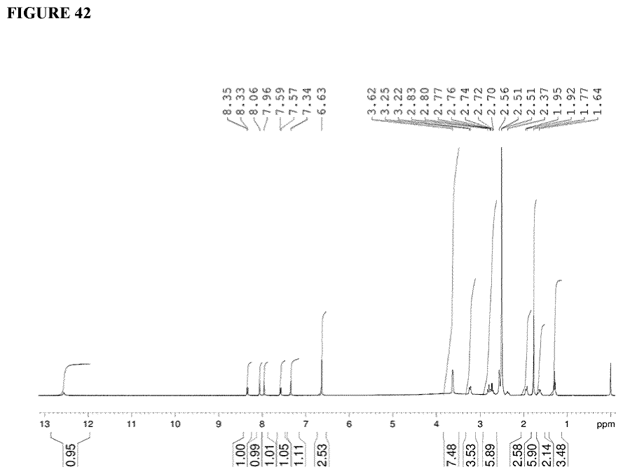

The solid Form 6 can be also characterized by XRPD pattern depicted in FIG. 34. The solid form can be further characterized by DSC pattern depicted in FIG. 35. The solid form can be further characterized by TGA pattern depicted in FIG. 36. The solid form can be also characterized by NMR spectrum depicted in FIG. 42.

The solid Form 6 of Alectinib fumaric acid (1:1) salt can be prepared by a process comprising:
a. Suspending solid Form 2 of Alectinib fumaric acid (1:1) salt in water to obtain a mixture;
b. Stirring the mixture for between 5 and 15 hours at a temperature between 20° C. and 25° C.;
c. Adding of methyl ethyl ketone;
d. Stirring for between 1 and 3 days at a temperature between 20° C. and 25° C.;
e. Isolating the solid form.

The concentration of Alectinib in water can be between 0.08 g/ml and 0.2 g/ml. The volume ratio between water and methyl ethyl ketone can be between 1:0.8 and 1:1.5. Alectinib is mixed with water to obtain a mixture. The mixture is stirred at a temperature between 20° C. and 25° C. for between 5 and 15 hours. To the mixture methyl ethyl ketone is added. The mixture is stirred at a temperature between 20° C. and 25° C. for between 1 and 3 days. The mixture is filtered off and optionally dried to provide solid Form 6 of Alectinib fumaric acid (1:1) salt.

The invention also relates to a solid form of Alectinib fumaric acid (2:1) salt, Form 2.

The solid Form 2 can be characterized by XRPD pattern having 2θ values 7.4°, 23.0° and 23.3° degrees 2 theta (±0.2 degrees 2 theta). The solid Form 2 of Alectinib fumaric acid (2:1) salt can be also characterized by XRPD pattern having 2θ values 7.4°, 12.9°, 14.3°, 18.5°, 23.0° and 23.3° degrees 2 theta (±0.2 degrees 2 theta). The solid form can be further characterized by XRPD pattern described in the following table:

| Angle (2θ) | Intensity (%) |
|---|---|
| 7.4 | 100.0 |
| 7.5 | 29.9 |
| 8.2 | 10.2 |
| 8.4 | 11.8 |
| 9.3 | 4.3 |
| 9.9 | 21.6 |
| 10.8 | 4.9 |
| 11.4 | 5.3 |
| 12.0 | 14.8 |
| 12.2 | 16.0 |
| 12.9 | 20.5 |
| 13.4 | 8.6 |
| 13.7 | 10.2 |
| 14.3 | 27.0 |
| 14.7 | 13.7 |
| 14.8 | 10.9 |
| 15.2 | 22.1 |
| 15.4 | 9.9 |
| 15.7 | 5.3 |
| 16.6 | 5.5 |
| 16.9 | 5.5 |
| 17.3 | 6.3 |
| 17.6 | 7.0 |
| 18.1 | 22.2 |
| 18.5 | 28.9 |
| 18.9 | 27.5 |
| 19.7 | 18.3 |
| 20.0 | 22.9 |
| 20.7 | 12.7 |
| 21.4 | 11.7 |
| 22.1 | 13.4 |
| 22.3 | 9.4 |
| 23.0 | 69.1 |
| 23.3 | 45.5 |
| 23.8 | 14.5 |
| 24.0 | 22.6 |
| 24.3 | 14.6 |
| 24.6 | 12.5 |
| 24.9 | 10.5 |
| 25.0 | 11.4 |
| 25.4 | 9.0 |
| 25.9 | 22.8 |
| 26.5 | 8.7 |
| 27.0 | 11.1 |
| 28.3 | 4.8 |
| 28.8 | 7.3 |
| 29.8 | 9.6 |
| 30.2 | 9.4 |
| 30.5 | 10.9 |
| 32.1 | 4.0 |

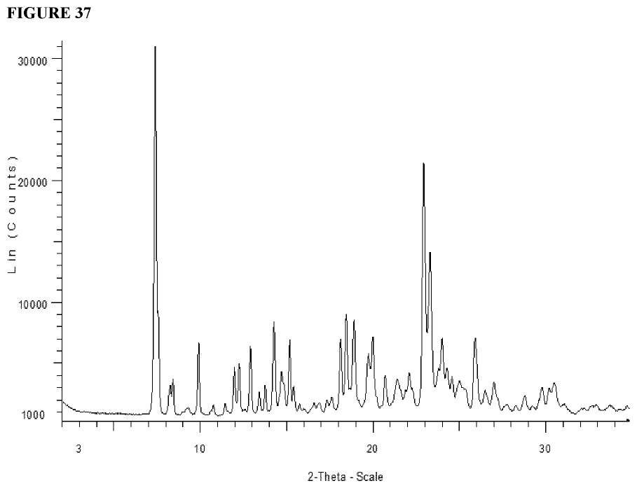

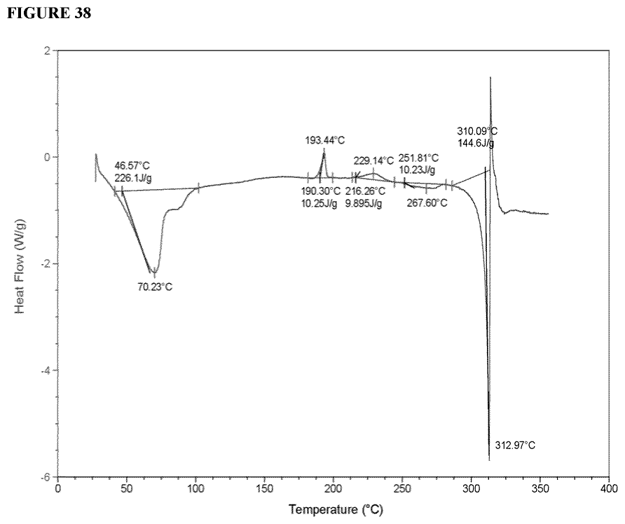

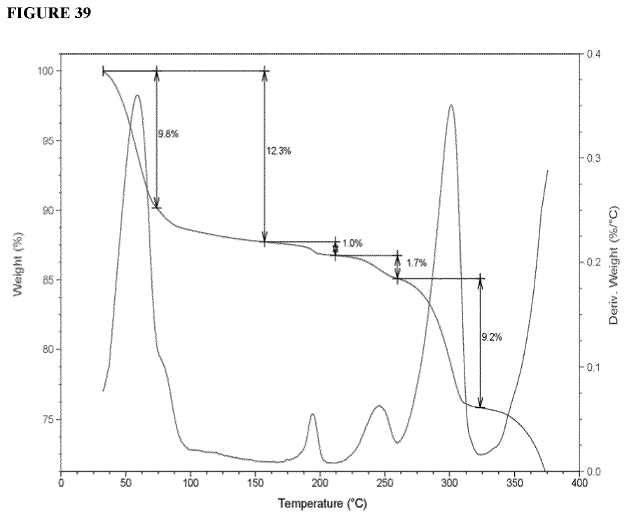

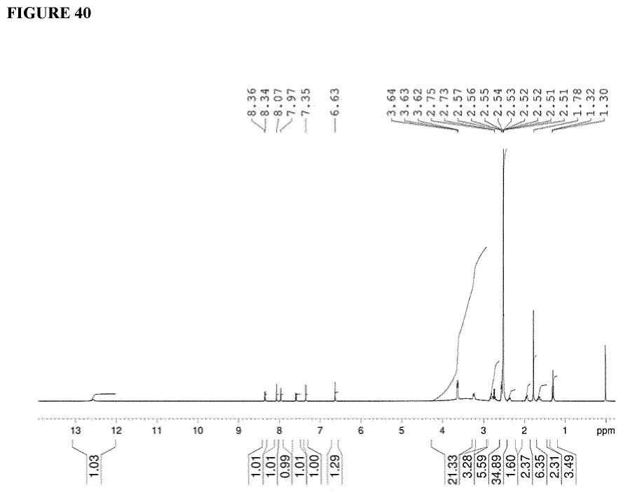

The solid Form 2 can be also characterized by XRPD pattern depicted in FIG. 37. The solid form can be further characterized by DSC pattern depicted in FIG. 38. The solid form can be further characterized by TGA pattern depicted in FIG. 39. The solid form can be also characterized by NMR spectrum depicted in FIG. 40.

The solid Form 2 of Alectinib fumaric acid (2:1) salt can be prepared by a process comprising:
a. Mixing fumaric acid with a mixture of 2-butanone, acetic acid and water to obtain a mixture;
b. Adding Alectinib to the mixture;
c. Isolating a solid form;
d. Drying the solid form on air for between 10 and 20 days.

The volume ratio between 2-butanone and water can be between 2.5:1 and 3.5:1. The volume ratio between 2-butanone and acetic acid can be between 5.5:1 and 6.5:1. The molar ratio between Alectinib and fumaric acid can be between 2:1 and 2.2:1. Fumaric acid is mixed with a mixture of 2-butanone, acetic acid and water to obtain a mixture. To the mixture Alectinib is added. Alectinib can be added either as solid or can be added in a solution with a suitable solvent. Obtained mixture is stirred at 20-25° C. for between 5 and 15 hours to obtain a suspension. The suspension is filtered to obtain a solid. The solid is dried on air for between and 20 days to obtain solid Form 2 of Alectinib fumaric acid (2:1) salt.

The presented invention also relates to Alectinib ethane sulfonate salt (esylate salt), solid forms thereof (Form 1, Form 2, Form 7) and processes for preparation thereof.

The solid Form 1 of Alectinib ethane sulfonate salt can be characterized by XRPD pattern having 2θ values 7.5°, 11.4°, 12.8° and 23.2° degrees 2 theta (±0.2 degrees 2 theta). The solid Form 1 can be also characterized by XRPD pattern having 2θ values 7.5°, 8.9°, 11.4°, 12.8°, 21.0° and 23.2° degrees 2 theta (±0.2 degrees 2 theta). The solid form can be further characterized by XRPD pattern described in the following table:

| Angle (2θ) | Intensity (%) |
|---|---|
| 7.5 | 41.9 |
| 8.4 | 36.8 |
| 8.9 | 53.6 |
| 9.9 | 19.7 |
| 10.8 | 20.0 |
| 11.4 | 49.6 |
| 11.6 | 7.6 |
| 12.8 | 89.5 |
| 13.2 | 12.4 |
| 13.8 | 5.4 |
| 14.2 | 38.5 |
| 14.9 | 30.6 |
| 15.7 | 23.8 |
| 16.3 | 8.5 |
| 17.0 | 24.0 |
| 17.5 | 15.7 |
| 17.8 | 19.5 |
| 18.0 | 15.4 |
| 18.4 | 42.7 |
| 18.8 | 10.7 |
| 19.9 | 38.8 |
| 20.6 | 13.0 |
| 21.0 | 67.6 |
| 21.6 | 19.0 |
| 21.8 | 16.7 |
| 22.5 | 26.6 |
| 22.8 | 81.8 |
| 23.2 | 100.0 |
| 23.5 | 19.6 |
| 24.1 | 8.8 |
| 24.4 | 7.0 |
| 24.9 | 16.3 |
| 25.5 | 8.8 |
| 25.8 | 10.3 |
| 26.1 | 7.7 |
| 26.5 | 17.1 |
| 26.8 | 16.6 |
| 27.0 | 18.2 |
| 27.3 | 8.6 |
| 27.9 | 8.2 |
| 28.6 | 8.7 |
| 28.9 | 5.3 |
| 29.5 | 29.6 |
| 29.8 | 14.3 |
| 30.3 | 15.3 |
| 31.1 | 7.5 |
| 31.8 | 5.3 |
| 32.2 | 4.9 |
| 32.5 | 10.0 |
| 32.8 | 7.0 |
| 33.2 | 6.1 |
| 33.7 | 4.1 |
| 34.0 | 4.7 |
| 34.5 | 8.0 |

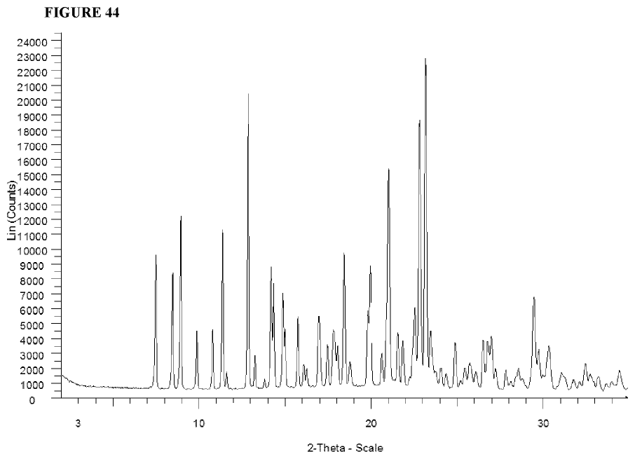

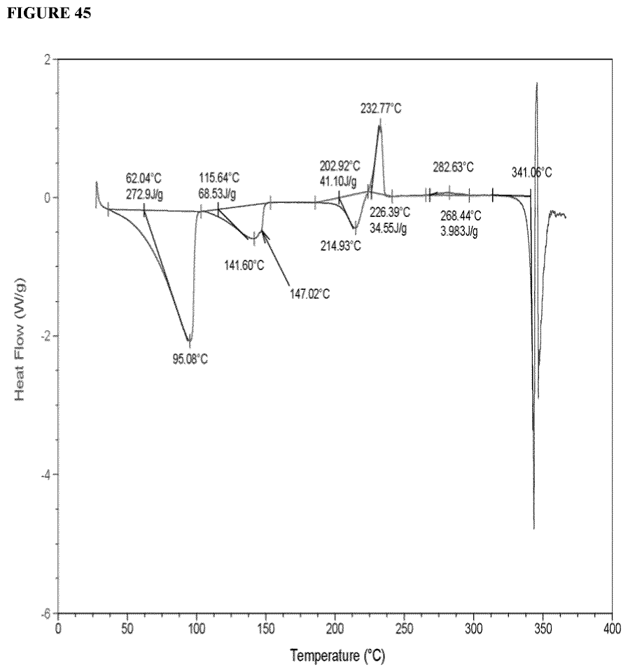

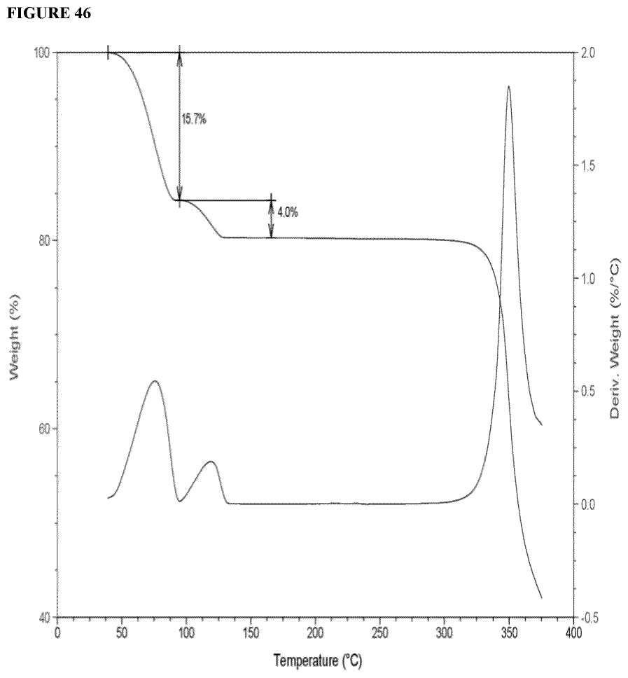

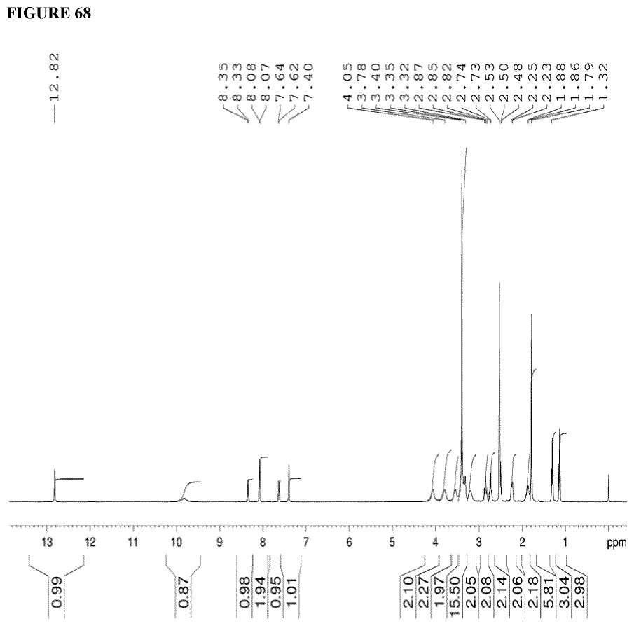

The solid Form 1 can be also characterized by XRPD pattern depicted in FIG. 44. The solid form can be further characterized by DSC pattern depicted in FIG. 45. The solid form can be further characterized by TGA pattern depicted in FIG. 46. The solid form can be also characterized by NMR spectrum depicted in FIG. 68.

The solid Form 1 of Alectinib ethane sulfonate salt can be prepared by a process comprising:
a. Mixing of Alectinib in mixture 2-butanone and water;
b. Adding of ethanesulfonic acid;
c. Isolating the solid Form 1.

The volume ratio between 2-butanone and water can be between 2.8:1 and 3.2:1. Concentration of Alectinib in the mixture of 2-butanone and water can be between 0.04 and 0.08 g/ml, preferably it is between 0.05 g/ml and 0.07 g/ml. The concentration of ethane sulfonic acid in the mixture of 2-butanone and water can be between 0.008 g/ml and 0.015 g/ml. The molar ratio between Alectinib and ethane sulfonic acid can be between 1:1 and 1.1:1. Alectinib is mixed with a mixture of 2-butanone and water. To the mixture ethane sulfonic acid is added. The mixture is stirred at room temperature (20-25° C.) for between 5 and 12 hours. The solid Form 1 is filtered off and optionally dried.

The solid Form 1 of Alectinib ethane sulfonate salt can be also prepared by a process comprising:
a. Mixing of Alectinib in mixture 2-butanone, acetic acid and water to obtain a mixture;
b. Adding the mixture to a mixture of ethanesulfonic acid and ethanol;
c. Isolating the solid Form 1.

The volume ratio between 2-butanone and acetic acid can be between 5.8:1 and 6.5:1. The volume ratio between 2-butanone and water can be between 2.8:1 and 3.5:1. The concentration of Alectinib in the mixture 2-butanone, acetic acid and water can be between 0.04 and 0.08 g/ml. The concentration of ethane sulfonic acid in ethanol can be between 0.006 g/ml and 0.01 g/ml. The molar ratio between Alectinib and ethane sulfonic acid can be between 1:1.1 and 1:1.4. Alectinib is mixed with the mixture of 2-butanone, acetic acid and water to obtain a mixture. The mixture is added to the mixture of ethane sulfonic acid in ethanol. The mixture is stirred at a temperature between 20° C. and 25° C. for between 10 and hours to obtain a suspension. The suspension is filtered off and obtained solid is dried in vacuum (10-15 kPa) at a temperature between 45° C. and 55° C. Obtained solid is left standing at a temperature between 20° C. and 25° C. for between 10 and 12 hours on air to provide solid Form 1 of Alectinib ethane sulfonate salt.

The solid Form 2 of Alectinib ethane sulfonate salt can be characterized by XRPD pattern having 2θ values 7.1°, 7.6°, 13.8° and 24.2° degrees 2 theta (±0.2 degrees 2 theta). The solid Form 2 can be also characterized by XRPD pattern having 2θ values 7.1°, 7.6°, 9.9°, 13.8°, 14.4° and 24.2° degrees 2 theta (±0.2 degrees 2 theta). The solid form can be further characterized by XRPD pattern described in the following table:

| Angle (2θ) | Intensity (%) |
|---|---|
| 7.1 | 58.4 |
| 7.6 | 70.4 |
| 7.8 | 42.0 |
| 9.4 | 27.2 |
| 9.9 | 74.5 |
| 11.0 | 35.6 |
| 11.6 | 15.8 |
| 12.5 | 41.1 |
| 12.7 | 21.4 |
| 12.9 | 19.3 |
| 13.4 | 43.9 |
| 13.8 | 65.4 |
| 14.2 | 43.0 |
| 14.4 | 56.2 |
| 15.2 | 16.1 |
| 15.7 | 21.5 |
| 16.2 | 11.5 |
| 17.2 | 12.7 |
| 18.0 | 40.1 |
| 18.9 | 39.3 |
| 19.4 | 50.6 |
| 19.8 | 71.3 |
| 20.2 | 69.3 |
| 21.1 | 18.6 |
| 22.2 | 37.2 |
| 22.8 | 21.2 |
| 23.3 | 46.7 |
| 24.2 | 100.0 |
| 24.6 | 50.2 |
| 27.2 | 18.9 |
| 27.7 | 17.6 |
| 28.4 | 10.0 |
| 28.9 | 12.9 |
| 29.8 | 7.7 |
| 30.0 | 8.8 |
| 30.4 | 10.6 |
| 31.6 | 14.8 |
| 32.1 | 9.4 |
| 32.7 | 9.2 |
| 33.1 | 11.5 |
| 34.6 | 5.7 |

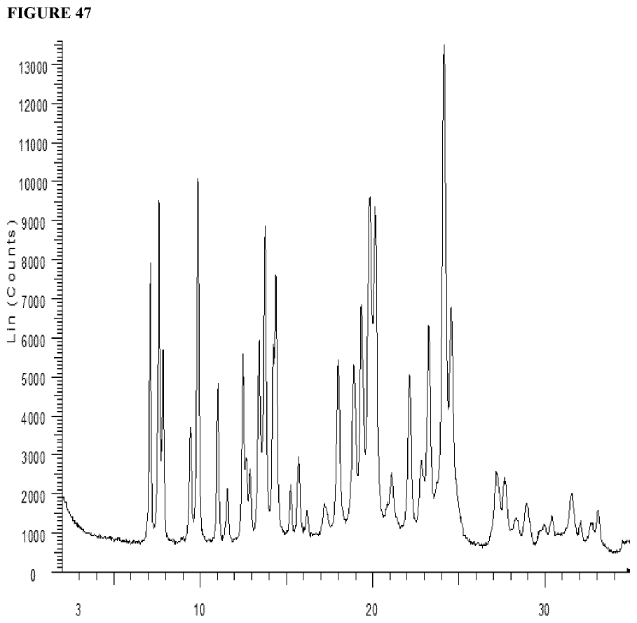

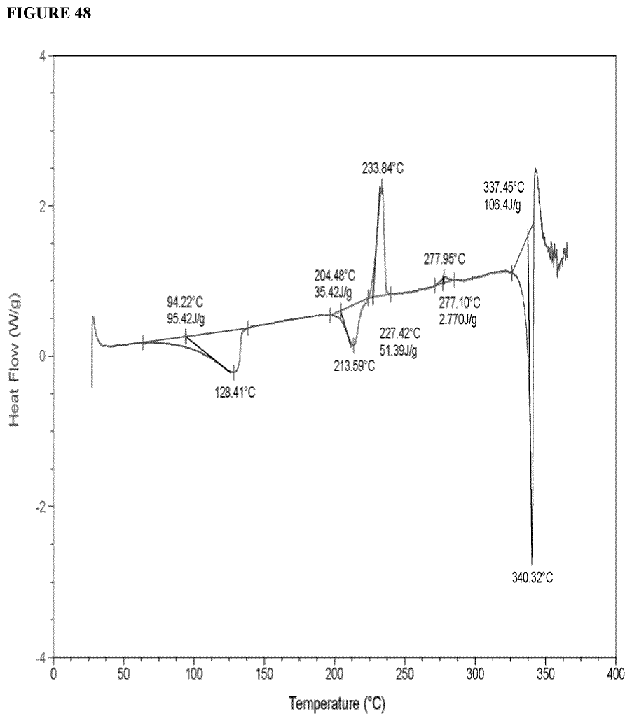

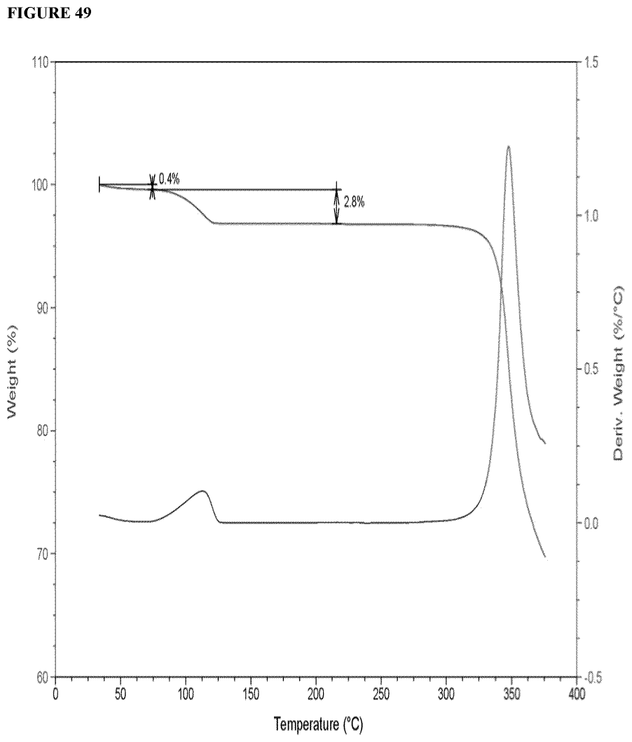

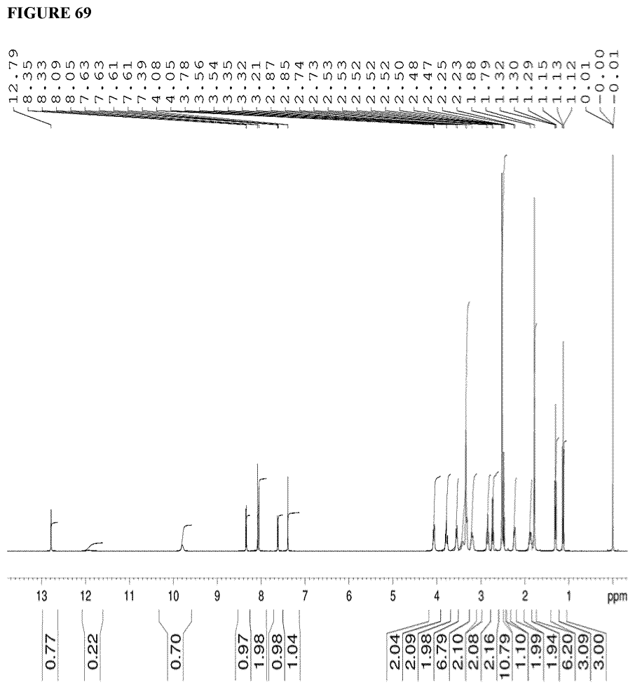

The solid Form 2 can be also characterized by XRPD pattern depicted in FIG. 47. The solid form can be further characterized by DSC pattern depicted in FIG. 48. The solid form can be further characterized by TGA pattern depicted in FIG. 49. The solid form can be also characterized by NMR spectrum depicted in FIG. 69.

The solid Form 2 of Alectinib ethane sulfonate salt can be prepared by a process comprising:
a. Mixing of Alectinib in mixture 2-butanone and water and acetic acid to obtain a mixture;
b. Adding the mixture into a solution of ethane sulfonic acid in ethanol;
c. Isolating a solid form of Alectinib;
d. Drying the solid form to obtain solid Form 2.

The volume ratio between 2-butanone and water can be between 2.5:1 and 3.5:1, preferably it is between 2.7:1 and 3:1. The volume ratio between water and acetic acid can be between 1.5:1 and 2.2:1, preferably it is between 1.7:1 and 2:1. The concentration of Alectinib in the mixture of 2-butanone and water and acetic acid can be between 0.03 g/ml and 0.07 g/ml, preferably it is between 0.04 g/ml and 0.06 g/ml. The concentration of ethane sulfonic acid in ethanol can be between 0.005 g/ml and 0.01 g/ml, preferably it is between 0.007 g/ml and 0.009 g/ml. The volume ratio between ethanol and the mixture of 2-butanone and water and acetic acid can be between 1.4:1 and 1.8:1, preferably it is between 1.5:1 and 1.7:1. Alectinib is mixed with the mixture of 2-butanone and water and acetic acid. Obtained mixture is added to the solution of ethane sulfonic acid in ethanol. The mixture is stirred at room temperature (20-25° C.) for between 1 and 3 days to obtain suspension. The solid is filtered off and dried, for example at room temperature under vacuum for between 8 and 12 hours to provide solid Form 2.

The solid Form 7 of Alectinib ethane sulfonate can be characterized by XRPD pattern having 2θ values 8.3°, 13.7°, 15.0 and 18.3° degrees 2 theta (±0.2 degrees 2 theta). The solid Form 7 can be also characterized by XRPD pattern having 2θ values 8.3°, 11.8°, 13.7°, 15.0, 5 18.3° and 20.2° degrees 2 theta (±0.2 degrees 2 theta). The solid Form 7 can be further characterized by XRPD pattern described in the following table:

| Angle (2θ) | Intensity (%) |
|---|---|
| 7.5 | 3.9 |
| 8.3 | 55.2 |
| 8.9 | 3.3 |
| 9.1 | 7.5 |
| 9.9 | 1.9 |
| 10.3 | 2.5 |
| 10.6 | 13.7 |
| 10.9 | 13.8 |
| 11.2 | 11.5 |
| 11.8 | 25.7 |
| 12.7 | 5.9 |
| 12.9 | 4.6 |
| 13.5 | 11.3 |
| 13.7 | 60.2 |
| 14.2 | 3.2 |
| 14.4 | 4.4 |
| 15.0 | 67.3 |
| 15.5 | 4.9 |
| 15.8 | 15.3 |
| 16.3 | 3.5 |
| 16.7 | 12.7 |
| 17.2 | 7.1 |
| 17.6 | 18.8 |
| 18.3 | 100.0 |
| 18.9 | 3.4 |
| 19.3 | 12.0 |
| 19.8 | 11.5 |
| 20.2 | 29.3 |
| 20.6 | 8.1 |
| 20.9 | 17.9 |
| 21.3 | 14.9 |
| 21.9 | 14.6 |
| 22.1 | 5.9 |
| 22.5 | 20.5 |
| 22.9 | 8.7 |
| 23.1 | 13.9 |
| 23.8 | 9.1 |
| 24.4 | 18.1 |
| 25.4 | 8.3 |
| 26.1 | 5.4 |
| 26.5 | 2.4 |
| 27.0 | 4.4 |
| 27.2 | 8.5 |
| 27.6 | 6.5 |
| 27.9 | 4.7 |
| 28.2 | 5.9 |
| 28.9 | 3.7 |
| 29.5 | 2.8 |
| 30.2 | 4.3 |
| 30.8 | 5.5 |
| 31.3 | 3.0 |
| 32.4 | 3.3 |
| 33.0 | 3.7 |
| 33.9 | 3.4 |
| 34.7 | 2.2 |

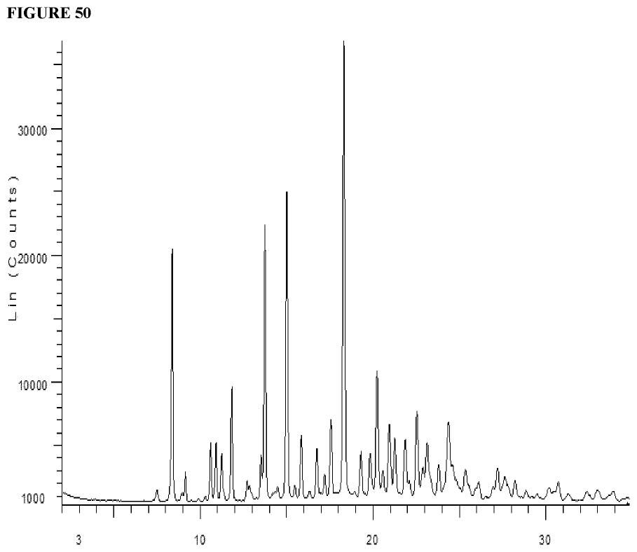

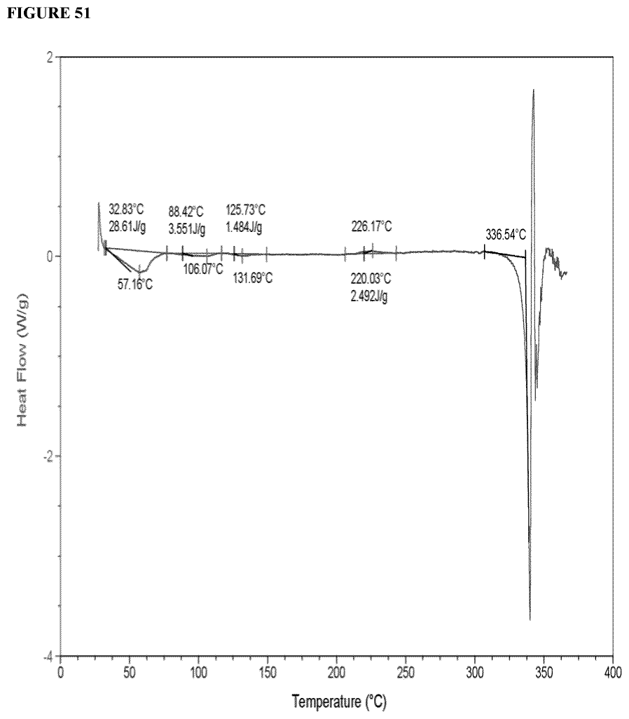

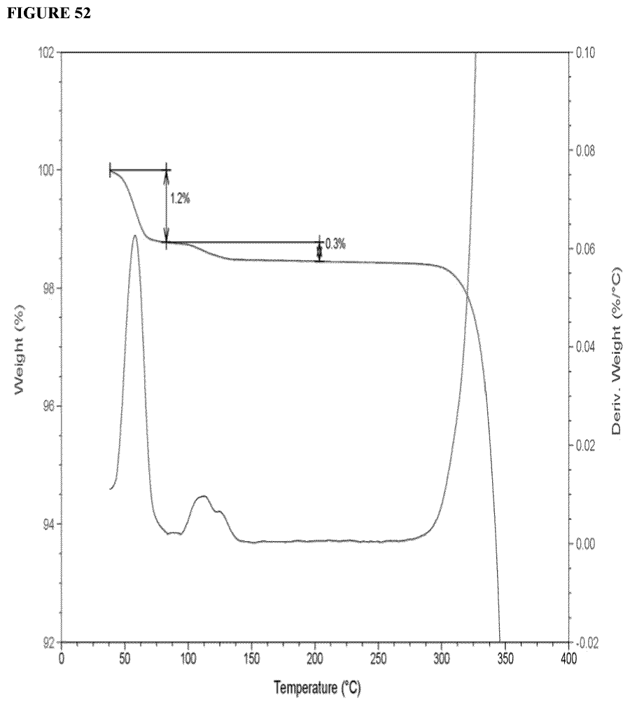

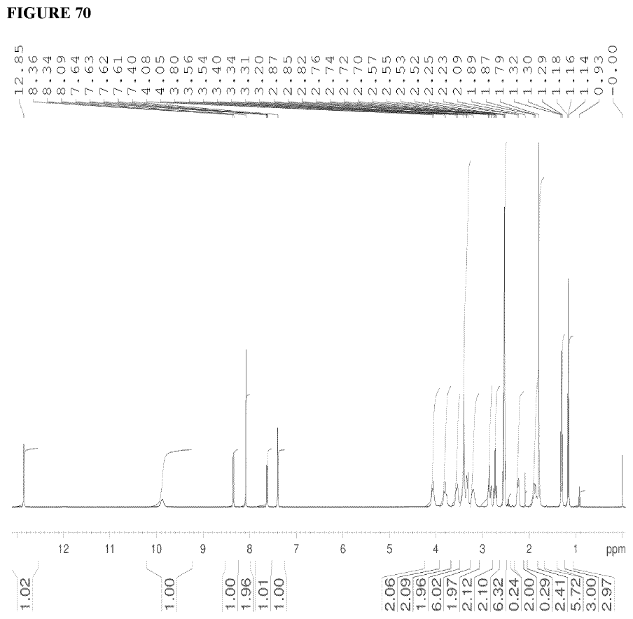

The solid Form 7 can be also characterized by XRPD pattern depicted in FIG. 50. The solid form can be further characterized by DSC pattern depicted in FIG. 51. The solid form can be further characterized by TGA pattern depicted in FIG. 52. The solid form can be also characterized by NMR spectrum depicted in FIG. 70.

The solid Form 7 of Alectinib ethane sulfonate salt can be prepared by a process comprising:
a. Suspending solid Form 1 of Alectinib ethane sulfonate, Form 1, in methyl ethyl ketone or ethanol for between 5 and 12 hours;
b. Isolating a solid;
c. Drying the solid on air for between 2 and 5 days.

The concentration of Alectinib ethane sulfonate Form 1 in methyl ethyl ketone can be between 0.08 g/ml and 0.25 g/ml. The mixture of Alectinib ethane sulfonate Form 1 in methyl ethyl ketone is stirred for between 5 and 12 hours at 20-25° C. The suspension is filtered and obtained solid is dried at 20-25° C. on air for between 2 and 5 days to obtain solid Form 7 of Alectinib ethane sulfonate salt.

The invention further relates to Alectinib methane sulfonate salt (mesylate salt), solid forms thereof (Form 3, Form 12, Form 13, Form 14, Form 15) and processes for preparing thereof.

The solid Form 3 of Alectinib methane sulfonate salt can be characterized by XRPD pattern having 2θ values 7.7°, 8.9°, 13.0° and 22.5° degrees 2 theta (±0.2 degrees 2 theta). The solid Form 3 can be also characterized by XRPD pattern having 2θ values 7.7°, 8.9°, 13.0°, 16.8°, 18.5°, 22.5° and 23.2° degrees 2 theta (±0.2 degrees 2 theta). The solid form can be further characterized by XRPD pattern described in the following table:

| Angle (2θ) | Intensity (%) |
|---|---|
| 7.1 | 15.1 |
| 7.7 | 96.4 |
| 8.4 | 28.9 |
| 8.9 | 64.2 |
| 9.2 | 14.9 |
| 10.0 | 14.2 |
| 10.8 | 23.3 |
| 11.2 | 39.8 |
| 11.8 | 10.3 |
| 13.0 | 82.5 |
| 13.2 | 27.8 |
| 14.1 | 19.6 |
| 14.2 | 24.6 |
| 14.7 | 22.7 |
| 15.0 | 32.2 |
| 15.3 | 15.8 |
| 15.8 | 24.9 |
| 16.2 | 13.9 |
| 16.8 | 40.4 |
| 17.8 | 30.0 |
| 18.0 | 40.8 |
| 18.3 | 17.3 |
| 18.5 | 53.3 |
| 18.9 | 8.1 |
| 19.8 | 17.0 |
| 20.1 | 23.9 |
| 20.6 | 42.0 |
| 21.0 | 17.9 |
| 21.5 | 48.1 |
| 21.7 | 24.9 |
| 22.2 | 20.0 |
| 22.5 | 100.0 |
| 23.2 | 84.6 |
| 23.6 | 27.2 |
| 24.4 | 25.8 |
| 24.9 | 7.0 |
| 25.3 | 8.0 |
| 25.8 | 10.7 |
| 26.2 | 17.2 |
| 26.6 | 18.5 |
| 26.8 | 20.2 |
| 27.3 | 8.2 |
| 27.9 | 7.9 |
| 28.3 | 10.3 |
| 28.7 | 9.8 |
| 29.4 | 25.9 |
| 29.8 | 20.8 |
| 30.4 | 11.9 |
| 31.0 | 9.7 |
| 31.6 | 7.4 |
| 32.1 | 8.5 |
| 32.5 | 5.7 |
| 32.8 | 7.8 |
| 33.3 | 5.0 |
| 33.8 | 8.2 |
| 34.2 | 7.9 |

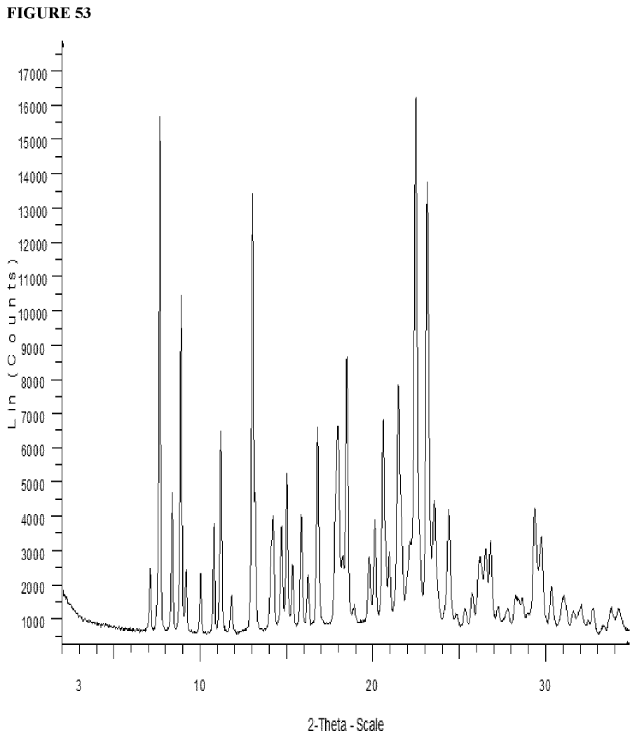

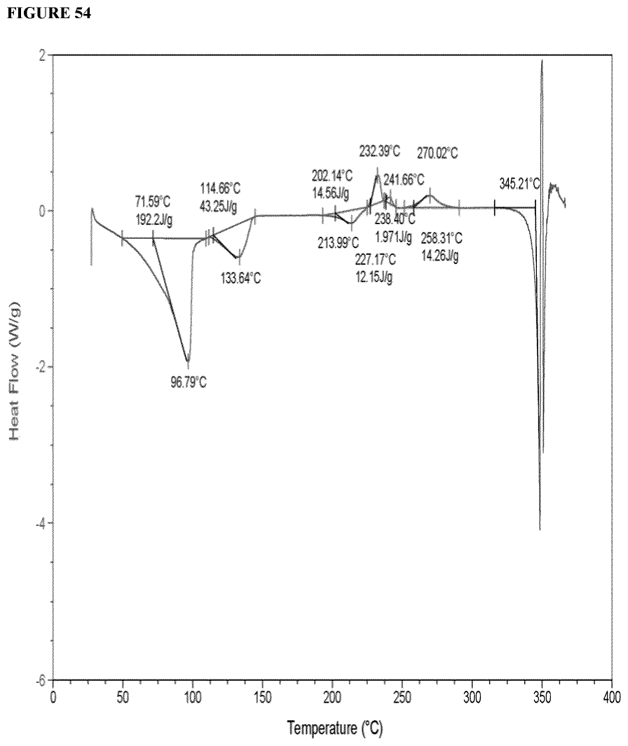

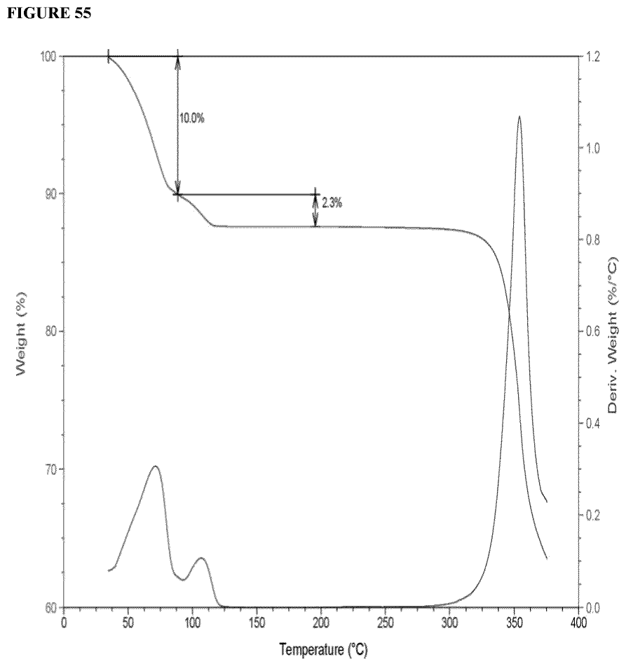

The solid Form 3 can be also characterized by XRPD pattern depicted in FIG. 53. The solid form can be further characterized by DSC pattern depicted in FIG. 54. The solid form can be further characterized by TGA pattern depicted in FIG. 55. The solid form can be also characterized by NMR spectrum depicted in FIG. 23.

The solid Form 3 of Alectinib methane sulfonate salt can be prepared by a process comprising:
a. Mixing Alectinib with a mixture of 2-butanone, water and acetic acid;
b. Adding of methane sulfonic acid;
c. Isolating a solid form of Alectinib methane sulfonic acid salt;
d. Contacting the solid form obtained in step c. at 20-25° C. with relative humidity between 30% and 80%.

The concentration of Alectinib in the mixture of 2-butanone, water and acetic acid can be between 0.04 g/ml and 0.07 g/ml. Volume ratio between 2-butanone and water can be between 2.5:1 and 3:1. Volume ratio between 2-butanone and acetic acid can be between 4.5:1 and 5.5:1. Molar ratio between Alectinib and methane sulfonic acid can be between 1:1 and 1:1.1. Methane sulfonic acid can be optionally added in a form of a solution in a suitable solvent, for example water. Alectinib is mixed with the mixture of 2-butanone, water and acetic acid. To the mixture methane sulfonic acid is added. The mixture is stirred at room temperature (20-25° C.) for between 15 and 24 hours to obtain a suspension. The suspension is filtered off to obtain a solid Alectinib salt with methane sulfonic acid. Obtained solid is contacted at 20-25° C. with relative humidity between 30% and 80% for between 1 and 12 hours to obtain solid Form 3 of Alectinib methane sulfonate salt.

The solid Form 12 of Alectinib methane sulfonate salt can be characterized by XRPD pattern having 2θ values 8.7°, 12.7°, 18.2° and 18.9° degrees 2 theta (±0.2 degrees 2 theta).

The solid Form 12 can be also characterized by XRPD pattern having 2θ values 8.7°, 11.1°, 12.7°, 18.2°, 18.9° and 19.7°degrees 2 theta (±0.2 degrees 2 theta). The solid form can be further characterized by XRPD pattern described in the following table:

| Angle (2θ) | Intensity (%) |
|---|---|
| 6.3 | 25.3 |
| 8.7 | 50.1 |
| 9.0 | 23.1 |
| 11.1 | 47.7 |
| 11.9 | 32.7 |
| 12.7 | 93.4 |
| 14.9 | 28.8 |
| 15.8 | 30.6 |
| 16.8 | 26.7 |
| 17.1 | 29.6 |
| 17.4 | 30.0 |
| 18.2 | 68.9 |
| 18.9 | 100.0 |
| 19.4 | 75.7 |
| 19.7 | 84.3 |
| 20.3 | 82.0 |
| 20.5 | 88.5 |
| 22.5 | 31.3 |
| 23.7 | 30.9 |
| 24.2 | 59.2 |
| 25.6 | 25.1 |
| 26.2 | 24.5 |
| 28.0 | 26.8 |
| 29.1 | 23.6 |

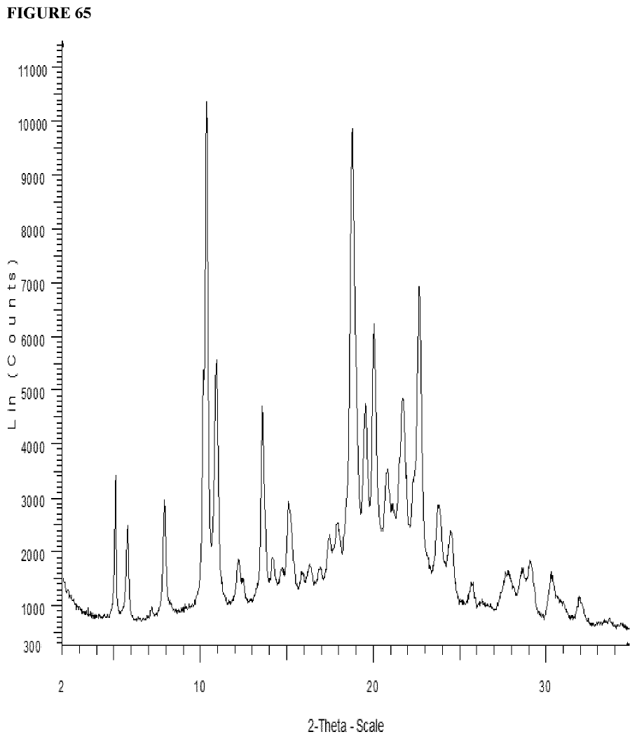

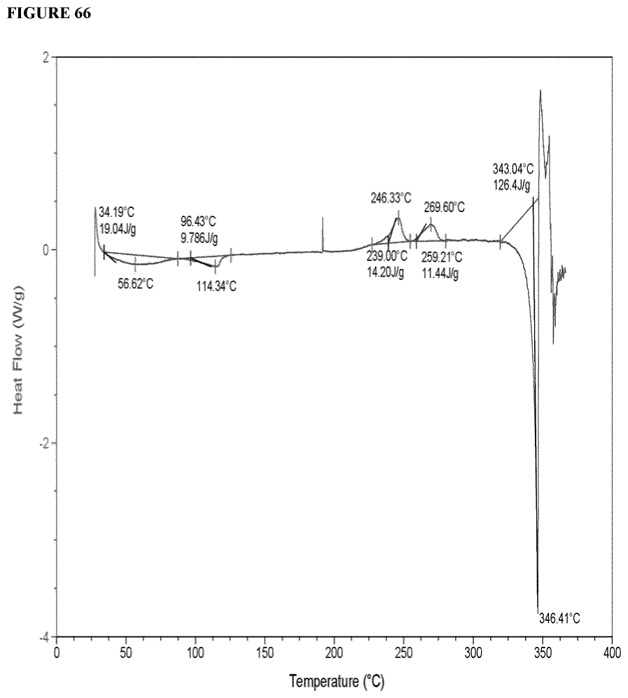

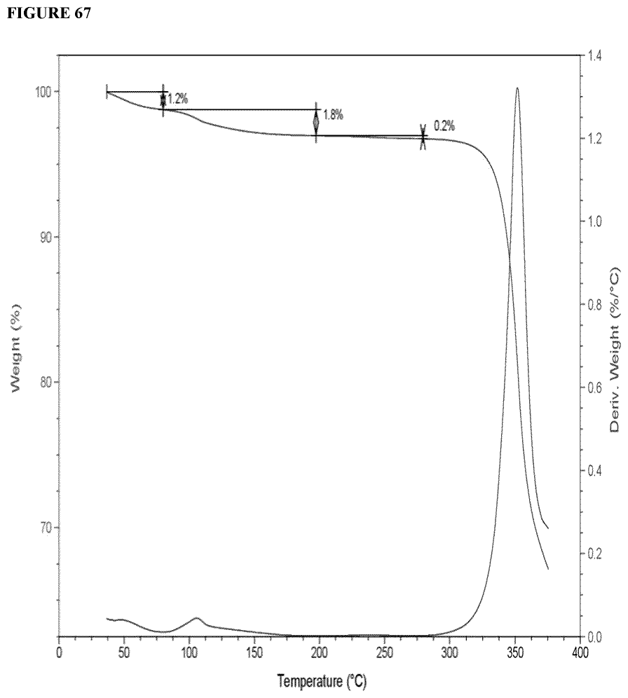

The solid Form 15 can be also characterized by XRPD pattern depicted in FIG. 65. The solid form can be further characterized by DSC pattern depicted in FIG. 66. The solid form can be further characterized by TGA pattern depicted in FIG. 67. The solid form can be also characterized by NMR spectrum depicted in FIG. 26.

The solid Form 12 of Alectinib methane sulfonate salt can be prepared by a process comprising:
a. Mixing Alectinib with a mixture of 2-butanone, water and acetic acid;
b. Adding of methane sulfonic acid;
C. Isolating a solid form of Alectinib methane sulfonic acid salt.

The concentration of Alectinib in the mixture of 2-butanone, water and acetic acid can be between 0.04 g/ml and 0.07 g/ml. The volume ratio between 2-butanone and water can be between 2.5:1 and 3.5:1. The volume ratio between 2-butanone and acetic acid can be between 5:1 and 5.5:1. The molar ration between Alectinib and methane sulfonic acid can be between 1:1 and 1:1.3. Methane sulfonic acid can be optionally added in a form of a solution in a suitable solvent. Alectinib is mixed with the mixture of 2-butanone, water and acetic acid. To the mixture methane sulfonic acid is added. The mixture is stirred at room temperature (20-25° C.) for between 2 and 6 hours to obtain a suspension. The suspension is filtered off, optionally washed with ethanol and obtained solid is immediately dried under vacuum (1-kPa) at 20-25° C. for between 2 and 15 hours to obtain solid Form 12 of Alectinib methane sulfonate salt.

The solid Form 13 of Alectinib methane sulfonate salt can be characterized by XRPD pattern having 2θ values 8.5°, 15.2° and 18.6° degrees 2 theta (±0.2 degrees 2 theta). The solid Form 13 can be also characterized by XRPD pattern having 2θ values 8.5°, 10.8°, 15.2°, 18.6° and 20.8° degrees 2 theta (±0.2 degrees 2 theta). The solid form can be further characterized by XRPD pattern described in the following table:

| Angle (2θ) | Intensity (%) |
|---|---|
| 2.2 | 6.1 |
| 7.8 | 4.9 |
| 8.5 | 59.1 |
| 9.3 | 5.2 |
| 10.8 | 27.5 |
| 11.3 | 10.7 |
| 12.0 | 22.9 |
| 12.8 | 10.3 |
| 13.2 | 5.3 |
| 14.0 | 36.7 |
| 15.2 | 100.0 |
| 15.7 | 7.3 |
| 15.9 | 10.6 |
| 16.7 | 15.9 |
| 17.1 | 7.8 |
| 17.3 | 12.4 |
| 17.6 | 17.4 |
| 17.8 | 11.6 |
| 18.6 | 61.6 |
| 19.2 | 4.7 |
| 19.9 | 15.2 |
| 20.8 | 32.3 |
| 21.4 | 20.8 |
| 21.7 | 19.6 |
| 22.1 | 10.0 |
| 22.4 | 18.7 |
| 23.3 | 25.8 |
| 23.9 | 15.9 |
| 24.7 | 26.3 |
| 25.2 | 22.3 |
| 25.8 | 9.1 |
| 26.5 | 7.9 |
| 26.8 | 5.7 |
| 27.4 | 3.8 |
| 28.0 | 10.2 |
| 28.5 | 6.4 |
| 29.5 | 5.4 |
| 30.1 | 3.5 |
| 30.9 | 3.3 |
| 31.4 | 6.0 |
| 32.2 | 3.0 |
| 32.9 | 3.2 |
| 33.3 | 3.1 |
| 34.0 | 4.3 |
| 34.5 | 3.3 |

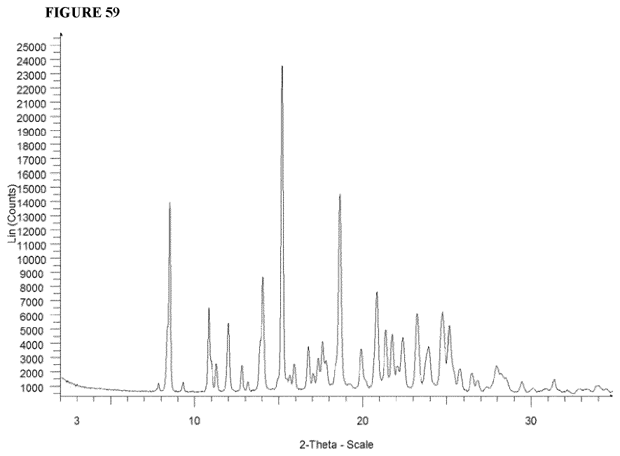

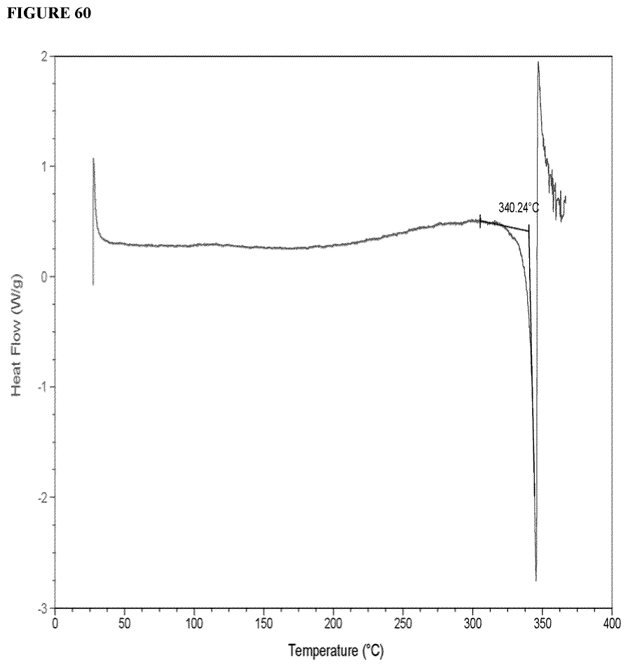

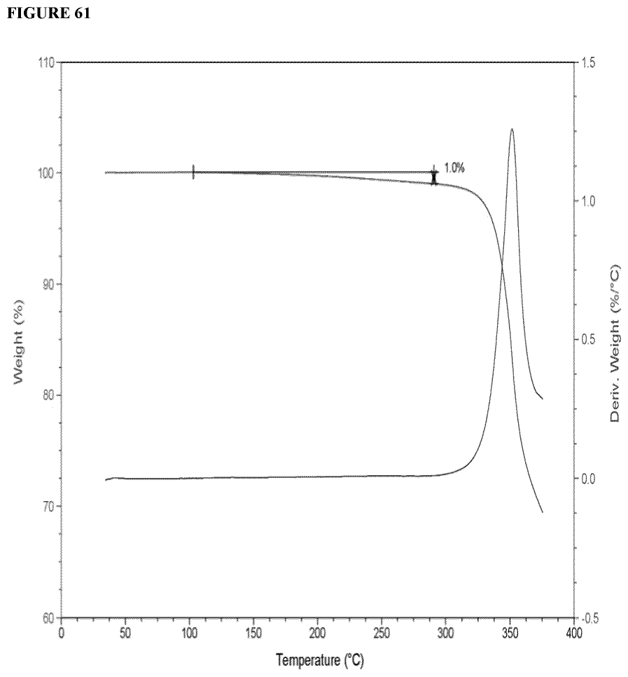

The solid Form 13 can be also characterized by XRPD pattern depicted in FIG. 59. The solid form can be further characterized by DSC pattern depicted in FIG. 60. The solid form can be further characterized by TGA pattern depicted in FIG. 61.

The solid Form 13 of Alectinib methane sulfonate salt can be prepared by a process comprising:
a. Suspending solid Form 3 of Alectinib methane sulfonate salt in 2-butanone;
b. Stirring the suspension for between 30 and 240 minutes;
c. Isolating a solid form of Alectinib methane sulfonic acid salt.

The concentration of solid Form 3 of Alectinib methane sulfonate salt in 2-butanone can be between 0.04 and 0.1 g/ml. The suspension is stirred for between 30 and 240 minutes at 20-25° C. The suspension is filtered off and obtained solid Form 13 of Alectinib methane sulfonate salt can be dried for example under vacuum at 20-25° C.

The solid Form 14 of Alectinib methane sulfonate salt can be characterized by XRPD pattern having 2θ values 5.7°, 7.9°, 10.8° and 12.1° degrees 2 theta (±0.2 degrees 2 theta). The solid Form 14 can be also characterized by XRPD pattern having 2θ values 5.7°, 7.9°, 10.8°, 12.1°, 18.9°, 20.2° and 21.7° degrees 2 theta (±0.2 degrees 2 theta). The solid form can be further characterized by XRPD pattern described in the following table:

| Angle (2θ) | Intensity (%) |
|---|---|
| 5.7 | 88.4 |
| 7.9 | 100.0 |
| 10.8 | 78.7 |
| 11.3 | 5.8 |
| 12.1 | 63.5 |
| 14.5 | 7.0 |
| 14.6 | 30.4 |
| 15.8 | 13.6 |
| 16.1 | 9.5 |
| 16.3 | 27.7 |
| 17.4 | 41.8 |
| 17.5 | 28.5 |
| 18.3 | 32.0 |
| 18.5 | 21.3 |
| 18.7 | 31.7 |
| 18.9 | 78.7 |
| 19.4 | 32.1 |
| 19.6 | 10.3 |
| 20.2 | 69.4 |
| 20.7 | 11.6 |
| 20.9 | 10.5 |
| 21.2 | 18.4 |
| 21.7 | 79.6 |
| 21.8 | 34.0 |
| 22.0 | 10.7 |
| 22.3 | 31.0 |
| 22.8 | 8.9 |
| 23.5 | 11.3 |
| 23.7 | 20.2 |
| 24.2 | 14.7 |
| 24.4 | 15.3 |
| 24.7 | 6.4 |
| 25.5 | 8.5 |
| 26.1 | 9.3 |
| 26.4 | 5.3 |
| 26.6 | 7.7 |
| 27.1 | 5.7 |
| 27.5 | 5.5 |
| 27.9 | 7.0 |
| 28.2 | 13.0 |
| 28.5 | 20.8 |
| 29.1 | 23.6 |
| 30.1 | 7.3 |
| 30.2 | 6.9 |
| 30.4 | 6.2 |
| 30.7 | 3.4 |
| 31.0 | 3.5 |
| 32.2 | 3.9 |
| 32.5 | 4.6 |
| 33.0 | 5.3 |
| 33.6 | 2.5 |

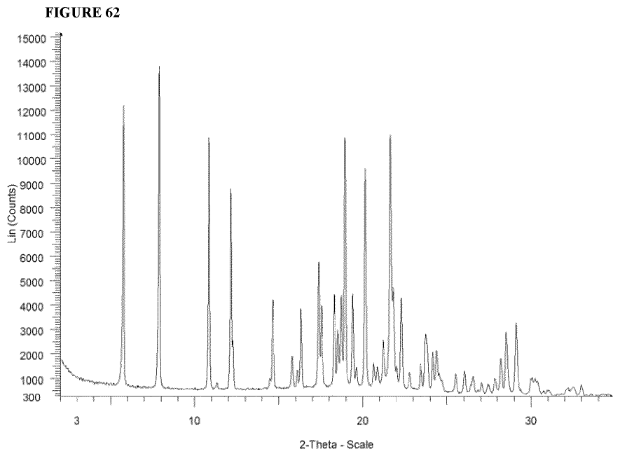

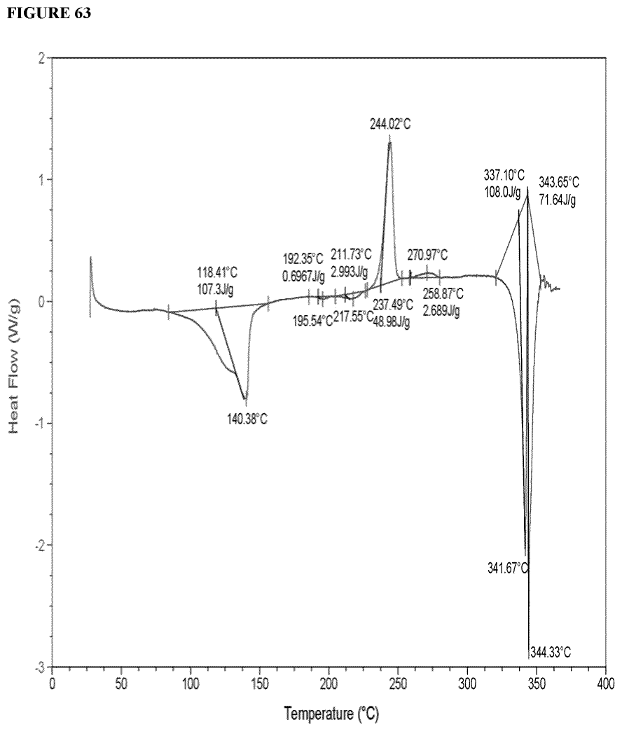

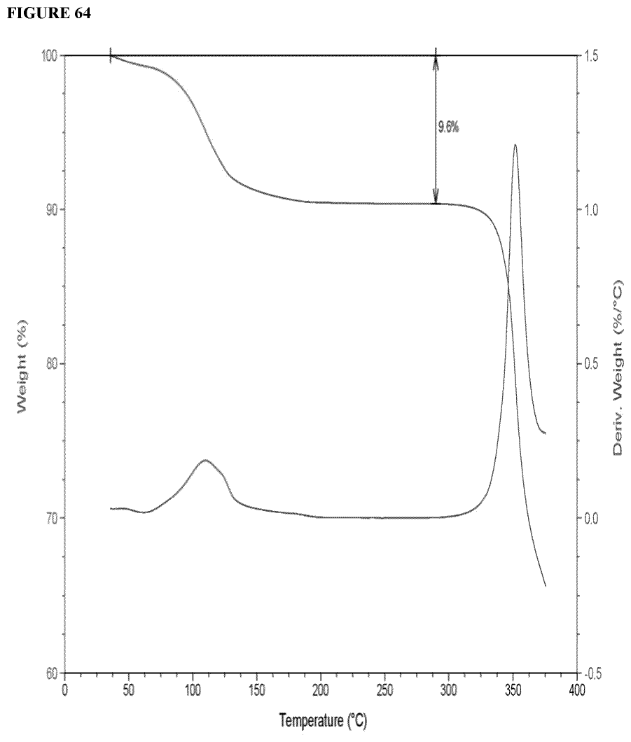

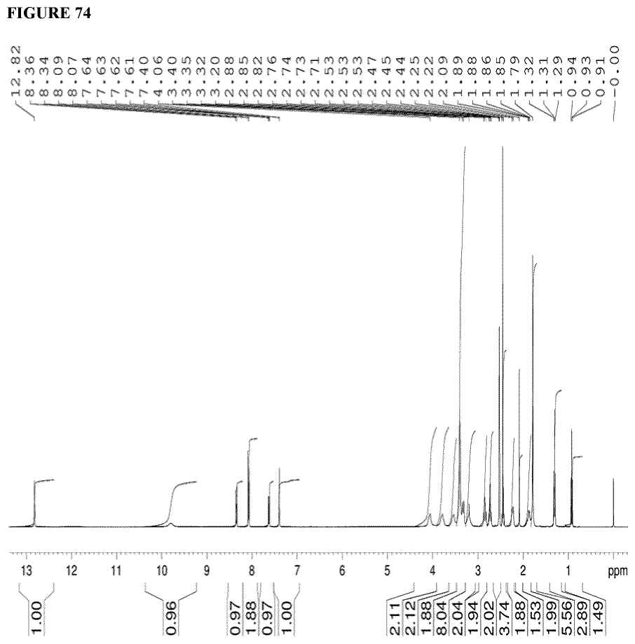

The solid Form 14 can be also characterized by XRPD pattern depicted in FIG. 62. The solid form can be further characterized by DSC pattern depicted in FIG. 63. The solid form can be further characterized by TGA pattern depicted in FIG. 64. The solid form can be also characterized by NMR spectrum depicted in FIG. 74.

The solid Form 14 of Alectinib methane sulfonate salt can be prepared by a process comprising:
a. Suspending solid Form 3 of Alectinib methane sulfonate salt in 2-butanone;
b. Stirring the suspension for between 30 and 240 minutes;
c. Isolating a solid form of Alectinib methane sulfonate salt.

The concentration of solid Form 3 of Alectinib methane sulfonate salt in 2-butanone can be between 0.07 g/ml and 0.15 g/ml. The mixture is stirred at 20-25° C. for between 60 and 240 minutes. The suspension is filtered off and obtained solid can be optionally dried at 20-25° C. on air.

The solid Form 15 of Alectinib methane sulfonate salt can be characterized by XRPD pattern having 2θ values 5.1°, 10.3°, 10.9° and 18.8° degrees 2 theta (±0.2 degrees 2 theta). The solid Form 15 can be also characterized by XRPD pattern having 2θ values 5.1°, 10.3°, 10.9°, 13.6°, 18.8°, 19.5°, 20.1° and 22.7° degrees 2 theta (±0.2 degrees 2 theta). The solid form can be further characterized by XRPD pattern described in the following table:

| Angle (2θ) | Intensity (%) |
|---|---|
| 5.1 | 32.8 |
| 5.8 | 23.8 |
| 7.2 | 9.3 |
| 7.9 | 28.3 |
| 10.3 | 100.0 |
| 10.9 | 53.7 |
| 12.2 | 17.9 |
| 12.4 | 14.3 |
| 13.6 | 45.2 |
| 14.2 | 17.9 |
| 14.7 | 16.2 |
| 15.1 | 28.2 |
| 15.9 | 15.5 |
| 16.3 | 16.8 |
| 16.9 | 16.5 |
| 17.5 | 22.3 |
| 17.9 | 24.2 |
| 18.8 | 95.1 |
| 19.5 | 45.7 |
| 20.1 | 59.9 |
| 20.8 | 33.8 |
| 21.2 | 27.9 |
| 21.7 | 46.6 |
| 22.3 | 32.4 |
| 22.7 | 66.9 |
| 23.8 | 27.6 |
| 24.5 | 22.9 |
| 25.8 | 13.5 |
| 26.3 | 10.7 |
| 27.8 | 15.6 |
| 28.7 | 16.2 |
| 29.1 | 17.6 |
| 30.3 | 15.4 |
| 31.0 | 10.3 |
| 32.0 | 10.9 |
| 33.7 | 7.2 |
| 34.4 | 6.3 |

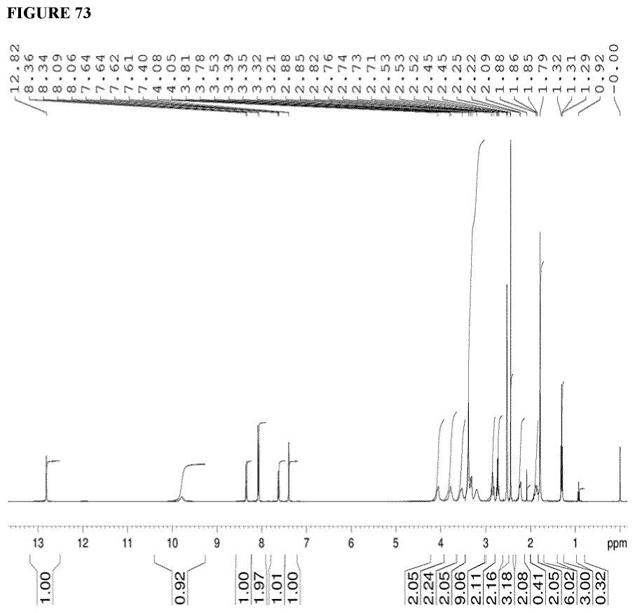

The solid Form 15 can be also characterized by XRPD pattern depicted in FIG. 65. The solid form can be further characterized by DSC pattern depicted in FIG. 66. The solid form can be further characterized by TGA pattern depicted in FIG. 67. The solid form can be also characterized by NMR spectrum depicted in FIG. 73.

The solid Form 15 of Alectinib methane sulfonate salt can be prepared by a process comprising drying Form 14 of Alectinib methane sulfonate salt in vacuum (1-10 kPa) for between 10 and 20 hours.

The invention will be further described with reference to the following examples.

### Examples

Nuclear magnetic resonance spectroscopy (NMR) was performed using Avance III 400 MHz NMR spectrometer.

DCS patterns were obtained using the following conditions: 10° C./min->350° C.

TGA patterns were obtained using the following conditions: 10° C./min->375° C.

XRPD spectrum was obtained using the following measurement conditions:

Panalytical Empyrean diffractometer with Θ/2Θ geometry (transmition mode), equipped with a PixCell 3D detector;

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406 Å (Kα1) | |
| Divergence slit: | ½° | |
| Antiscatter slit: | ½° | |
| Soller slit: | 0.02 | rad |
| Detector slit: | 7.5 | mm |
| Rotation speed: | 30 | rpm |

### Example 1: Alectinib Acetate Salt, Form 2

1 g of Alectinib was mixed with 12 ml of 2-butatonone and 2.4 ml of acetic acid. The mixture was stirred at 20-25° C. for 0.5 hour. The mixture was filtered. The filter cake was washed with 5 ml 2-butatonone to obtain a solid. The solid was dried for 6 days in vacuum drier (1-10 kPa) at 25° C. to provide solid Form 2 of Alectinib acetate salt in 80% yield.

### Example 2: Alectinib Acetate Salt, Forms 3, 4, 5

2 g of Alectinib was mixed with 24 ml of 2-butanone and 4 ml of acetic acid. The mixture was sonicated for 5 minutes and then stirred at 20-25° C. for 30 minutes. The solid in suspension was XRPD analyzed. It was solid Form 5 of Alectinib acetate. The mixture was stirred overnight (for 12 hours) and was filtered. Obtained solid was dried on air for 12 hours at 20-25° C. to provide solid form 4 of Alectinib acetate in almost 100% yield. In the case solid Form 5 of Alectinib acetate is dried in vacuum drier for 12 hours at 20-25° C., solid Form 3 of Alectinib acetate is obtained.

### Example 3: Alectinib Acetate Salt, Form 6

1 g of Alectinib was mixed with 12 ml of 2-butatonone and 2.4 ml of acetic acid. The mixture was stirred at 20-25° C. for 0.5 hour. The mixture was sonicated for 2 minutes. The mixture was heated to 50° C. for 15 minutes. The mixture was filtered, the filter cake was dried on open dish for 5 days to provide Alectinib acetate salt, Form 6 in 81% yield.

### Example 4: Alectinib Acetate Salt, Form 7

1 g of Alectinib was mixed with 12 ml of 2-butatonone and 24 ml of acetic acid. The mixture was heated to 50° C. and stirred at this temperature for 30 minutes. The mixture was cooled to 25° C. in the course of 2 hours. Obtained suspension was filtered. Obtained solid was dried at vacuum (10-15 kPa) at 25° C. for 12 h to provide solid Alectinib acetate salt, Form 7 in 93% yield.

### Example 5 Solid Form III of Alectinib

0.2 g of Alectinib free base was suspended in 20 ml acetone and the mixture heated to reflux. The mixture was stirred at reflux for 5 minutes. The mixture was filtered while hot. The clear filtrate was refluxed for a 10 minutes. The mixture was cooled to 20-25° C. and stirred at 20-25° C. for 2 hours. The mixture was filtered off. The obtained solid was air dried at 20-25° C. to provide solid Form III of Alectinib.

### Example 6: Solid Form III of Alectinib

0.2 g of Alectinib free base was suspended in 40 ml acetonitrile and heated to reflux.

The mixture was stirred at reflux for 2.5 hours. The suspension was cooled to 20-25° C. The suspension was filtered off and obtained solid was air-dried at 20-25° C. to provide solid Form III of Alectinib.

### Example 7: Solid Form IV of Alectinib

0.2 g of Alectinib free base was suspended in 28 ml 2-propanol and heated to reflux for 5 minutes. The mixture was cooled to 20-25° C. and stirred at this temperature for 15-30 minutes. Obtained suspension was filtered off and obtained solid was air-dried at 20-25° C. to provide solid Alectinib Form IV.

### Example 8: Solid Form V of Alectinib

0.2 g of Alectinib was mixed with 5 ml of methanol. The mixture was heated to 125-130° C. The solution was cooled to 20-25° C. Obtained suspension was filtered off. Obtained solid was air-dried at 20-25° C. to provide solid Form V of Alectinib.

### Example 9: Solid Form VI of Alectinib

0.2 g of Alectinib free base was mixed with 35 ml 1,4-dioxane. The mixture was heated to reflux and stirred at this temperature for 5 minutes. The mixture was cooled to 20-25° C. The suspension was filtered. Obtained solid was air dried at 20-25° C. to provide solid Form VI of Alectinib.

### Example 10: Solubilities of Prepared Salts

In the following table solubilities of preparad salts and prior art HCl salt (prepared according to WO2015163447) and tosylate salt (prepared according to CZ31293) are compared.

| Salt | Solubility after 1 hour (mg/ml) |
|---|---|
| HCl | 0.37 |
| Tosylate | 0.5 |
| Acetate, Form 4 | 1.22 |
| Acetate, Form 2 | 0.87 |

20 mg of the Alectinib salt was mixed 10 ml of a mixture of buffer pH 1.2 and 4% Triton X-100. The mixture was placed into a stirring carousel at temperature +37° C. and 100 rpm. After 1 hour and 4 hours about 1 mL of each sample was taken and filtered through a 0.45 μm filter into a vial. Then each sample was diluted with Solvent solution to get signal of absorbance ≤1 at 265 nm, transferred into a vial and measured by HPLC method.

It can be concluded, that solubilities of prepared acetate salts of Alectinib are improved comparing to prior art HCl or tosylate salts.

### Example 11: Alectinib Tartrate Salt, Form 8

3 g of Alectinib and 1.03 g of L-(+)-tartaric acid were mixed with 20 ml of water (20 ml). The suspension was stirred at 20-25° C. for 1.5 hour. The suspension was filtered off. Obtained solid was dried on filter over weekend at 20-25° C. to obtain a solid form of Alectinib tartrarte salt. Obtained solid was slurried for 12 hours in 4 ml of acetone. The mixture was filtered off, obtained solid was dried over weekend on air to provide 3.5 g of solid Form 8 of Alectinib tartrate.

### Example 12: Alectinib Hydrogen Maleate Salt, Form 1

2 g of Alectinib was dissolved in a mixture of 24 ml of 2-butatonone, 4 ml of acetic acid and 7 ml of water. To the mixture a solution of 0.48 g of maleic acid in 1 ml of water was added. The mixture was stirred at 20-25° C. overnight. Obtained suspension was filtered. The filter cake was washed with 5 ml 2-butanone and dried on ait at 20-25° C. for 12 hours to provide solid Form 1 of Alectinib hydrogen maleate

### Example 13: Alectinib Hydrogen Maleate Salt, Form 2

Alectinib hydrogen maleate salt obtained according to Example 8 was dried for 12 h in vacuum drier at 25° C. to provide solid Form 2 of Alectinib hydrogen maleate

### Example 14: Alectinib Hydrogen Maleate Salt, Form 2

2 g of Alectinib was mixed with a mixture of 24 ml of 2-butatonone, 4 ml of acetic acid and 7 ml of water. The mixture was seeded with 60 mg of Alectinib hydrogen maleate, Form 2. To the mixture 0.5 g of maleic acid was added. The mixture was stirred at 0-5° C. for 2 hours and filtered. The filter cake was dried on air for 2 days to provide 2.16 g of Alectinib hydrogen maleate, Form 2.

### Example 15: Alectinib fumaric acid (1:1) salt, Form 2

2 g of Alectinib was mixed with a mixture of 24 ml of 2-butatonone (24 ml), 7.2 ml of acetic acid and 8.4 ml of water. It was dropped during 5 minutes into a solution of 0.49 g of fumaric acid in 60 ml of ethanol and 4 ml of water. The mixture was stirred at 20-25° C. for 10 hours. The mixture was filtered and obtained solid was dried on air for 12 hours. Obtained solid was mixed with 20 ml of ethanol. The mixture was heated to 70° C. and stirred at this temperature for 0.5 hour. The mixture was stirred at 20-25° C. for 2 days. The mixture was filtered, the filter cake was rinsed with 5 ml of ethanol and obtained solid was dried for 12 hours on air to provide 1.6 g of solid Form 2 of Alectinib fumaric acid (1:1) salt.

### Example 16: Alectinib fumaric acid (1:1) salt, Form 6

2.48 g of solid Form 2 of Alectinib fumaric acid (1:1) salt was mixed with 20 ml of water. The mixture was stirred for 10 hours. To the mixture 20 ml of methyl ethyl ketone was added. The mixture was stirred for 2 days. The mixture was filtered. Obtained solid was washed with 5 ml of ethanol and dried on filter to provide 2.33 g of solid Form 6 of Alectinib fumaric acid (1:1) salt.

### Example 17: Alectinib Fumaric Acid (2:1) Salt, Form 2

0.24 g of fumaric acid was mixed with a mixture of 24 ml of 2-butatonone, 4 ml of acetic acid and 8 ml of water. To the mixture 2 g of Alectinib were added. The mixture was stirred at 20-25° C. for 12 hours. Obtained suspension was filtered. Filter cake was washed with 5 ml 2-butanone to provide a solid form of Alectinib fumaric acid (2:1) salt. Obtained solid form was dried on ait for 14 days to provide solid Form 2 of Alectinib fumaric acid (2:1) salt.

### Example 18: Solubilities of Prepared Salts

In the following table solubilities of preparades salts and prior art HCl salt (prepared according to WO2015163447) and tosylate salt (prepared according to CZ31293) are compared.

| Salt | Solubility after 1 hour (mg/ml) |
|---|---|
| HCl | 0.37 |
| Tosylate | 0.5 |
| Tartrate, Form 8 | 1.23 |
| Hydrogen maleate salt, Form 1 | 2.38 |
| Hydrogen maleate salt, Form 2 | 1.26 |
| Fumaric acid (1:1) salt, Form 2 | 1.30 |
| Fumaric acid (1:1) salt, Form 6 | 1.22 |
| Fumaric acid (2:1) salt, Form 2 | 1.23 |

20 mg of the Alectinib salt was mixed 10 ml of a mixture of buffer pH 1.2 and 4% Triton X-100. The mixture was placed into a stirring carousel at temperature+37° C. and 100 rpm. After 1 hour and 4 hours about 1 mL of each sample was taken and filtered through a 0.45 μm filter into a vial. Then each sample was diluted with Solvent solution to get signal of absorbance≤1 at 265 nm, transferred into a vial and measured by HPLC method. It can be concluded, that solubilities of prepared tartrate, hydrogen maleate, fumarate (1:2 and 2:1) salts are improved comparing to prior art HCl or tosylate salts.

### Example 19: Alectinib Ethane Sulfonate Salt, Form 1

2 g of Alectinib were mixed with 24 ml of 2-butanone and 8 ml of water. To the mixture 0.47 g of ethanesulfonic acid were added. The mixture was stirred for 10 hours at 20-25° C. and filtered. The filter cake was dried at 20-25° C. for 3 hours on air to provide 2.56 g of Form 1 of Alectinib ethane sulfonate salt.

### Example 20: Alectinib Ethane Sulfonate Salt, Form 2

1 f of Alectinib was mixed with a mixture of 12 ml of 2-butatonone, 2.4 ml of acetic acid and 4.3 ml of water. The mixture was poured into a solution of 0.24 g of ethanesulfonic acid in 30 ml of ethanol. The mixture was stirred at 20-25° C. over weekend. The mixture was filtered and the filter cake was dried in vacuum drier (1-10 kPa) at 25° C. overnight to provide 0.61 mg of Form 2 of Alectinib ethane sulfonate salt.

### Example 21: Alectinib Ethane Sulfonate Salt, Form 7

3 g of Alectinib was diluted in 36 ml of 2-butatonone, 7 ml of acetic acid and 12 mol of water. Obtained mixture was poured into solution of 0.71 g of ethane sulfonic acid in 90 ml of ethanol. The mixture was stirred at 20-25° C. for 5 hours. The mixture was filtered to obtain a solid. Obtained solid was dried on air overnight (for 12 hours) and then dried at vacuum drier (1-10 kPa) for 12 hours overnight. Obtained solid was suspended in 10 ml of ethanol and the suspension was stirred for 10 hours. The suspension was filtered and dried for 3 hours on air to provide solid Alectinib ethane sulfonate salt, Form 7 in 65% yield.

### Example 22: Alectinib Ethane Sulfonate Salt, Form 7

1.27 g of Alectinib ethane sulfonate, Form 1, was suspended in 10 ml of methyl ethyl ketone and the suspension was stirred for 10 hours. The suspension was filtered off, obtained solid was dried for 3 hours in vacuum (1-10 kPa). Obtained solid was stored over weekend on air to provide Alectinib ethane sulfonate salt, Form 7 in 91% yield.

### Example 23: Alectinib Methane Sulfonate Salt, Form 3

3 g of Alectinib was mixed with a mixture of 36 ml of 2-butatonone, 7.2 ml of acetic acid and 13 ml of water. To the mixture 0.63 f of methane sulfonic acid was added. The mixture was stirred for 20 hours at ambient temperature (20-25° C.). The suspension was filtered. The filter cake was washed with 15 ml ethanol sucked on filter for 30 minutes. Obtained solid was dried on air at 20-25° C. and relative humidity between 30% and 80% for 4 hours to provide Form 3 of Alectinib methane sulfonate salt in yield 79%.

### Example 24: Alectinib Methane Sulfonate Salt, Form 12

1 g od Alectinib was mixed with a mixture of 12 ml of 2-butanone, 2.3 ml of acetic acid and 4 ml of water. To the mixture 225 mg of methanesulfonic acid was added. The mixture was stirred at 20-25° C. for 4 hours, filtered and the filter cake was washed with 4 ml of 2-butanone. Obtained solid was dried in vacuum drier (25° C., 130 mbar, N2 bleed, 12 hours) to provide 0.82 g of Alectinib methane sulfonate salt, Form 12.

### Example 25: Alectinib Methane Sulfonate Salt, Form 13

1.11 g of solid Form 3 of Alectinib methane sulfonate salt was mixed with 20 ml of 2-butanone. The mixture was stirred for 1 hour. Obtained suspension was filtered and the filter cake was dried in vacuum drier (25° C., 130 mbar, N2 bleed, 1 hour) to provide 0.93 g of solid Form 13 of Alectinib mesylate salt.

### Example 26: Alectinib Methane Sulfonate Salt, Form 14 and 15

2.76 g of solid Form 3 of Alectinib methane sulfonate salt was mixed with 28 ml of 2-butanone. The mixture was stirred for 1.5 hours. The suspension was filtered, sucked for 15 minutes on filter to provide solid Form 14 of Alectinib methane sulfonate salt. Obtained solid Form 14 was dried in vacuum (25° C., 130 mbar, N2 bleed, 15 hour) to provide 2.4 g of solid Form 15 of Alectinib methane sulfonate salt.

### Example 27: Alectinib Ethane Sulfonate Salt, Form 1

2 g of Alectinib was mixed with a mixture of 24 ml of 2-butanone, 4 ml of acetic acid and 8 ml of water. Obtained mixture was poured into a solution of 0.47 g of ethane sulfonic acid in 60 ml of ethanol. The mixture was stirred at 20-25° C. for 12 hours. Obtained suspension was filtered and the filter cake was dried 17 h in vacuum at 50° C. The solid was then left standing for 12 hours on air to provide 1.22 g of solid Form 1 of Alectinib ethane sulfonate salt.

### Example 28: Solubilities of Prepared Salts

In the following table solubilities of prepared salts and prior art HCl salt (prepared according to WO2015163447) and tosylate salt (prepared according to CZ31293) are compared.

| Salt | Solubility after 1 hour (mg/ml) |
|---|---|
| HCl | 0.37 |
| Tosylate | 0.5 |
| Ethane sulfonate salt, Form 1 | 1.27 |
| Ethane sulfonate salt, Form 2 | 1.24 |
| Ethane sulfonate salt, Form 7 | 1.25 |
| Methane sulfonate salt, Form 3 | 1.21 |
| Methane sulfonate salt, Form 12 | 1.35 |
| Methane sulfonate salt, Form 13 | 1.30 |
| Methane sulfonate salt, Form 14 | 1.32 |
| Methane sulfonate salt, Form 15 | 1.19 |

20 mg of the Alectinib salt was mixed 10 ml of a mixture of buffer pH 1.2 and 4% Triton X-100. The mixture was placed into a stirring carousel at temperature+37° C. and 100 rpm. After 1 hour and 4 hours about 1 mL of each sample was taken and filtered through a 0.45 μm filter into a vial. Then each sample was diluted with Solvent solution to get signal of absorbance≤1 at 265 nm, transferred into a vial and measured by HPLC method.

It can be concluded, that solubilities of prepared ethane sulfonate or methane sulfonate salts of Alectinib are improved comparing to prior art HCl or tosylate salts.

1-15. (canceled)

16. An Alectinib salt selected from the group consisting of an Alectinib acetate salt, an Alectinib salt with tartaric acid, an Alectinib salt with maleic acid, an Alectinib fumaric acid (1:1) salt, an Alectinib fumaric acid (2:1) salt, an Alectinib ethane sulfonate salt, and an Alectinib methane sulfonate salt.

17. The Alectinib salt according to claim 16, wherein said salt is an Alectinib acetate salt.

18. The Alectinib acetate salt according to claim 17, wherein said salt is in a solid form.

19. The solid form of Alectinib acetate salt according to claim 18, wherein said solid form is Form 2, which is characterized by XRPD pattern having 2θ values 10.7°, 11.0°, 13.1° and 19.9° degrees 2 theta (±0.2 degrees 2 theta).

20. The Alectinib salt according to claim 16, wherein said salt is an Alectinib salt with tartaric acid.

21. The Alectinib salt with tartaric acid according to claim 20, wherein said salt is in a solid form.

22. The Alectinib salt according to claim 16, wherein said salt in an Alectinib salt with maleic acid.

23. The Alectinib salt with maleic acid according to claim 22, wherein said salt is in a solid form.

24. The Alectinib salt according to claim 16, wherein said salt is an Alectinib fumaric acid (1:1) salt.

25. The Alectinib fumaric acid (1:1) salt according to claim 24, wherein said salt is in a solid form.

26. The Alectinib salt according to claim 16, wherein said salt is an Alectinib fumaric acid (2:1) salt.

27. The Alectinib fumaric acid (2:1) salt according to claim 26, wherein said salt is in a solid form.

28. The Alectinib salt according to claim 16, wherein said salt is an Alectinib ethane sulfonate salt.

29. The Alectinib ethane sulfonate salt according to claim 28, wherein said salt is in a solid form.

30. The Alectinib salt according to claim 16, wherein said salt is an Alectinib methane sulfonate salt.

31. The Alectinib methane sulfonate salt according to claim 30, wherein said salt is in a solid form.
